(19) United States
(12) Patent Application Publication
(10) Pub. No.: US 2021/0121503 A1
(43) Pub. Date: Apr. 29, 2021

(54) FECAL MICROBIOTA TRANSPLANTATION FOR TREATING ULCERATIVE COLITIS

(71) Applicant: CRESTOVO HOLDINGS LLC, Somerville, MA (US)

(72) Inventor: Thomas Julius BORODY, Five Dock (AU)

(73) Assignee: Crestovo Holdings LLC, Somerville, MA (US)

(21) Appl. No.: 16/755,794

(22) PCT Filed: Oct. 12, 2018
(86) PCT No.: PCT/US2018/055635

§ 371 Date: Apr. 13, 2020

Related U.S. Application Data
(60) Provisional application No. 62/571,620, filed on Oct. 12, 2017.

(51) Int. Cl. A61K 35/74; A61P 1/12; A61K 35/38

(52) U.S. Cl. CPC A61K 35/74; A61K 35/38; A61P 1/12

(57) ABSTRACT

The present disclosure provides methods and treatment regimens for treating an inflammatory bowel disease (IBD), e.g., ulcerative colitis (UC), in a subject in need thereof. The present disclosure further provides adjusting a dosing regimen for fecal-microbiome therapy of IBD based on a level of a fecal marker for intestinal inflammation. Further provided are methods comprising providing a therapeutic dosing regimen to a patient with a gastrointestinal disorder (e.g., IBD) in need thereof, the method comprising administering to the patient a therapeutic composition comprising fecal microbes based upon a level of a fecal marker for intestinal inflammation. An exemplary fecal marker is calprotectin. This disclosure also provides a method for screening or selecting a fecal donor by testing efficacy of the donor's fecal material in inducing desirable changes in a fecal marker for intestinal inflammation in an IBD patient (e.g., UC patient).

### CROSS-REFERENCE TO RELATED APPLICATION

This application claims priority to U.S. Provisional Application No. 62/571,620, filed Oct. 12, 2017, which is incorporated by reference in its entirety herein.

### FIELD

The present disclosure relates to methods and dosing regimens suitable for treating ulcerative colitis in a subject in need thereof.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and results in increased susceptibility to pathogens, altered metabolic profiles, or induction of proinflammatory signals that can lead to local or systemic inflammation or autoimmunity. The intestinal microbiota plays a significant role in the pathogenesis of many disorders such as pathogenic infections of the gut.

Ulcerative colitis is a chronic disease of the large intestine, also known as the colon, in which the lining of the colon becomes inflamed and develops tiny open sores, or ulcers, that produce pus and mucous. Ulcerative colitis occurs most often in people ages 15 to 30, although the disease may afflict people of any age. It affects men and women equally and appears to run in some families.

Ulcerative colitis is a disease that is characterized by inflammation and micro-ulcers in the superficial layers of the large intestine. The inflammation usually occurs in the rectum and lower part of the colon, but it may affect the entire large intestine (pancolitis). Ulcerative colitis can very rarely affect the small intestine in its distal portion (Backwash Ileitis).

The inflammation is accompanied usually with diarrhea, which may be profuse and bloody. Micro-ulcers form in places where inflammation has destroyed the cells lining the bowel and these areas bleed and produce pus and mucus. Ulcerative colitis, especially when mild, can be difficult to diagnose because symptoms are similar to other intestinal disorders, most notably the other type of Irritable Bowel Diseases (IBD) called Crohn's disease and also irritable bowel syndrome. Crohn's disease differs from ulcerative colitis because it causes inflammation throughout the whole thickness of the intestinal wall and produces deep ulcers. Crohn's disease usually occurs in the small intestine, but it can also occur in the large intestine, anus, esophagus, stomach, appendix and mouth. Crohn's disease causes fistulae whereas ulcerative colitis does not. Both Crohn's and ulcerative colitis may co-exist in the same patient. The combination of inflammation and ulceration can cause abdominal discomfort and frequent emptying of the colon. Existing treatments for ulcerative colitis involve intense and lengthy combinational drug therapy with significant side effects or even require surgery to remove part of the colon. Moreover, a substantial proportion of ulcerative colitis patients are resistant to standard drug therapy. Thus, there is a need for more effective treatments for ulcerative colitis that are easier to administer.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

Fecal microbiota transplantation (FMT), also known as ‘fecal bacteriotherapy,’ represents the one therapeutic protocol that allows the fastest reconstitution of a normal composition and functional gut microbial community. FMT has been offered by select centers across the world, typically as an option of last resort for patients with recurrent Clostridium difficile infection (CDI). FMT has also been suggested in treating other gut infective agents such as E. coli and Vancomycin resistant Enterococci (VRE). Currently, FMT is administered by several routes including infusion of human microbiota in the form of homogenized stool, extracts of homogenized stool, or cultured stool components through a colonoscope, an enema, or via a nasojejunal tube.

### SUMMARY

In one aspect, this disclosure provides a method of providing a therapeutic dosing regimen to a patient with a gastrointestinal (GI) disorder in need thereof, the method comprising administering to the patient a therapeutic composition comprising viable non-pathogenic fecal bacteria based upon a level of a fecal marker of intestinal inflammation, wherein the fecal marker comprises a protein secreted by an immune cell of the patient.

In another aspect, this disclosure provides a method for optimizing the dosing regimen of a fecal microbe-based therapy in a patient with a gastrointestinal disorder in need thereof, the method comprising:
a. administering to the patient a therapeutic composition comprising fecal microbes at a first dosing regimen comprising a first dosage at a first dosing frequency;
b. determining the level of a fecal marker for intestinal inflammation in the patient, wherein the fecal marker comprises a protein secreted by an immune cell of the patient; and
c. modifying the first dosing frequency based on the level of the fecal marker for intestinal inflammation.

In one aspect, this disclosure provides a method for screening a fecal donor, the method comprising administering to a test subject a fecal therapeutic composition derived from the fecal donor, measuring a fecal marker for intestinal inflammation in the test subject, wherein the fecal marker comprises a protein secreted by an immune cell of the test subject, and selecting the fecal donor based on the level of the fecal marker for intestinal inflammation.

### BRIEF DESCRIPTION OF THE DRAWINGS

FIG. 1 shows a study patient CONSORT Flow Diagram in accordance with Example 1 of the present disclosure;

FIG. 2 shows a graphical representation of the study design in accordance with Example 4 of the present disclosure;

FIG. 3A shows the number of patients in the FMT and placebo-treated groups achieving the primary outcome of steroid-free clinical remission and endoscopic remission or response at week 8 after therapy in accordance with Example 5 of the present disclosure;

FIG. 3B shows the number of patients in steroid-free clinical remission and clinical response at week 8 after therapy in accordance with the Examples of the present disclosure;

FIG. 3C shows the number of patients with steroid-free endoscopic response and complete mucosal healing after therapy in accordance with the Examples of the present disclosure;

FIG. 4A shows an exemplary baseline endoscopic appearance of 25 cm recto-sigmoid active colitis in accordance with Example 5 of the present disclosure;

FIG. 4B shows an exemplary endoscopic appearance at end of week 8 blinded FMT therapy in accordance with Example 5 of the present disclosure;

FIG. 4C shows an exemplary baseline endoscopic appearance of extensive colitis to the hepatic flexure in accordance with Example 5 of the present disclosure;

FIG. 4D shows an exemplary endoscopic appearance at the completion of 8 weeks open-label FMT in accordance with Example 5 of the present disclosure;

FIG. 5 shows the speed of onset of therapy in accordance with Example 5 of the present disclosure;

FIG. 6A shows the number of operational taxonomic units (OTUs) per fecal sample in accordance with Example 6 of the present disclosure;

FIG. 6B shows the phylogenetic diversity within each fecal sample in accordance with Example 6 of the present disclosure;

FIG. 6C shows the principal component analysis of fecal samples at the genus taxonomic level in accordance with Example 6 of the present disclosure;

FIG. 6D shows the number of OTUs in blinded study patients on FMT therapy, according to primary outcome, individual donors, and donor batches in accordance with Example 6 of the present disclosure.

FIG. 7A shows the endoscopy images of (left 1 and 2) marked UC inflammation of the rectum prior to treatment; (right 1) dramatic reduction in inflammation with stool attaching to the mucosa of the rectum at week 20; (left 3 and 4) marked UC inflammation in the sigmoid colon prior to treatment; and (right 2 and 3) marked reduction in inflammation in the sigmoid colon at week 20. Right 2 shows stool attached to the inflamed wall of the sigmoid colon. Right 3 shows scattered changes in inflammation.

FIG. 7B shows the endoscopy images of (left 7, 8, 9, and 10) inflammation and significant mucus in the traverse colon at week 8 post-treatment; and (right 6) improvement in inflammation at week 20. In right 6, the inflammation in the transverse colon is improved but still visible. The vessels are not visible.

### DETAILED DESCRIPTION

Before the present compositions and methods are described, it is to be understood that the present disclosure is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular aspects of the disclosure, and not to exhaustively specify all permutations, combinations and variations thereof.

Unless defined otherwise herein, terms are to be understood according to conventional usage by those of ordinary skill in the relevant art. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of 20%, 10%, 5%, 1%, 0.5%, or even 0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “substantially free” as used herein when referring to a substance's presence in a composition, is meant to encompass that the substance constitutes less than 1%, less than 0.5%, less than 0.1%, or even less than 0.01% of the whole substance by volume or mass.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, a “GI disorder” refer to a disease or disorder involving the gastrointestinal tract, namely the esophagus, stomach, small intestine, large intestine, and rectum, and the accessory organs of digestion, the liver, gallbladder, and pancreas.

As used herein, a “condition having a GI component” refers to a condition, disease, or disorder which has an either causal or correlative link with one or more GI functions or dysfunctions. Such condition may or may not manifest any common GI symptoms such as heartburn, indigestion/dyspepsia, bloating and constipation.

As used herein, “therapeutically effective amount” or “pharmaceutically active dose” refers to an amount of a composition which is effective in treating the named disease, disorder or condition.

As used herein, “microbiota,” and “flora” refer to a community of microbes that live in or on a subject's body, both sustainably and transiently, including eukaryotes, archaea, bacteria, and viruses (including bacterial viruses (i.e., phage)). A non-selective fecal microbiota refers to a community or mixture of fecal microbes derived from a donor's fecal sample without selection and substantially resembling microbial constituents and population structures found in such fecal sample.

As used herein, “remission,” “cure,” or “resolution rate” refers to the percentage of patients that are cured or obtain remission or complete resolution of a condition in response to a given treatment. As used herein, “clinical remission sustaining rate” refers to the percentage of patients remaining in clinical remission after a specified post-treatment period among all patients who achieve remission at the completion of a treatment. Quantitatively, remission, cure, or resolution is achieved when a patient's UCDAI score is below or equal to 2, assessed after 8 weeks of treatment. Remission, cure, or resolution can be further confirmed by endoscopic and mucosal healing.

As used herein “steroid-free” refers to a complete lack or a substantial lack of steroid use.

As used herein, “primary outcome rate” refers to the percentage of patients achieving primary outcome after a specific treatment or treatment regimen among all patients receiving that treatment or treatment regimen.

As used herein, “response rate” refers to the percentage of patients that respond positively to a given treatment. Quantitatively, a patient responds to a treatment positively when the patient's UCDAI score decreases by at least 2 from baseline to week 8.

As used herein, “Mayo Clinic score” or “Mayo score” refers to an index system for assessing the severity of a ulcerative colitis disease condition. See Table 1 and Schoeder et al. N Engl J Med 1987; 317:1625-9. The Mayo Clinic score ranges from 0-12, with sub-scores of 0-3, where the higher scores indicate more severe disease. In some aspects, sub-scores may be rated for stool frequency, rectal bleeding, mucosal appearance at endoscopy, and physician's global assessment (PGA).

TABLE 1
Mayo Clinic Scoring System for Assessment of
Ulcerative Colitis Activity (Shoeder et al.)

| | score |
|---|---|
| | assignment |
| 1. Stool frequency* | |
| Normal number of stools for this patient | 0 |
| 1-2 stools more than normal | 1 |
| 3-4 stools more than normal | 2 |
| 5 or more stools more than normal | 3 |
| 2. Rectal Bleeding† | |
| No blood seen | 0 |
| Streaks of blood with stool less half the time | 1 |
| Obvious blood with stool most of the time | 2 |
| Blood alone passed | 3 |
| 3. Findings of flexible proctosigmoidoscopy | |
| Normal or inactive disease | 0 |
| Mild disease (erythema, decreased vascular pattern, | 1 |
| mild friability) | |
| Moderate disease (marked erythema, absent vascular | 2 |
| pattern, friability, erosions) | |
| Severe disease (spontaneous bleeding, ulceration) | 3 |
| 4. Physician's global assessments‡ | |
| Normal | 0 |
| Mild disease | 1 |
| Moderate disease | 2 |
| Severe disease | 3 |

*Each patient served as his or her own control to establish the degree of abnormality of the stool frequency
†The daily bleeding score represented the most severe bleeding of the day
‡The physician's global assessment acknowledged the three other criteria, the patient's daily record of abdominal discomfort and general sense of well-being, and other observations, such as physical findings and the patient's performance status

As used herein, “ulcerative colitis endoscopic index of severity” or “UCEIS” refers to an index for assessing endoscopic disease activity. The index assesses three criteria, including vascular pattern, bleeding, and erosions and ulcers (Table 2). See Travis et al., Developing an instrument to assess the endoscopic severity of ulcerative colitis: the Ulcerative Colitis Endoscopic Index of Severity (UCEIS). A higher score reflects increased disease severity.

TABLE 2
Scoring System for Ulcerative Colitis Endoscopic
Index of Severity (See Travis et al.)

| | score |
|---|---|
| | assignment |
| 1. Vascular pattern | |
| Normal: Normal vascular pattern with arborization of | 1 |
| capillaries clearly defined, or with blurring or patchy loss of | |
| capillary margins | |
| Patchy obliteration: Patchy obliteration of vascular pattern | 2 |
| Obliterated: Complete obliteration of vascular pattern | 3 |
| 2. Rectal bleeding | |
| None: No visible blood | 1 |
| Mucosal: Some spots or streaks of coagulated blood on the | 2 |
| surface of the mucosa ahead of the scope, which can be | |
| washed away | |
| Luminal mild: Some free liquid blood in the lumen | 3 |
| Luminal moderate or severe: Frank blood in the lumen ahead | 4 |
| of endoscope or visible oozing from mucosa after washing | |
| intra-luminal blood, or visible oozing from a hemorrhagic | |
| mucosa | |
| 3. Erosions and ulcers | |
| None: Normal mucosa, nonvisible erosions or ulcers | 1 |
| Erosions: Tiny (≤5 mm) defects in the mucosa, of a white or | 2 |
| yellow color with a flat edge | |
| Superficial ulcer: Larger (>5 mm) defects in the mucosa, | 3 |
| which are discrete fibrin-covered ulcers when compared to | |
| erosions, but remain superficial | |
| Deep ulcer: Deeper excavated defects in the mucosa, with a | 4 |
| slightly raised edge | |

As used herein, “ulcerative colitis disease activity index” or “UCDAI” refers to an index system for assessing the symptomatic severity or response of a ulcerative colitis patient. The index assesses four variables, which include stool frequency, severity of bleeding, colonic mucosal appearance, and the physician's overall assessment of disease activity (Table 3). See Sutherland et al., 5-Aminosalicylic acid enema in the treatment of distal ulcerative colitis, proctosigmoiditis, and proctitis. Gastroenterology. 1987; 92:1894-8. Each variable is scored from 0-3 so that the total index score ranges from 0-12; 0-2: remission; 3-6: mild; 7-10: moderate; >10: severe ulcerative colitis.

TABLE 3
Scoring System for Ulcerative Colitis Disease
Activity Index. (See Tursi et al.)

| | score |
|---|---|
| | assignment |
| 1. Stool frequency | |
| Normal | 0 |
| 1-2 Stools/day > normal | 1 |
| 3-4 Stools/day > normal | 2 |
| >4 Stools/day > normal | 3 |
| 2. Rectal bleeding | |
| None | 0 |
| Streaks of blood | 1 |
| Obvious blood | 2 |
| Mostly blood | 3 |
| 3. Mucosal appearance | |
| Normal | 0 |
| Mild friability | 1 |
| Moderate friability | 2 |
| Exudation, spontaneous bleeding | 3 |
| 4. Physician's rating of disease activity | |
| Normal | 0 |
| Mild | 1 |
| Moderate | 2 |
| Severe | 3 |

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and lack organelles.

As used herein, “fecal bacteria” refers to bacteria that can be found in fecal matter.

As used herein, “viable” means possessing the ability to multiply.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is the Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “operational taxonomic unit” or “OTU” refers to a group of closely related microbial species determined based on 16S or 18S rRNA marker genes.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

Different types of ulcerative colitis exist. As used herein, “ulcerative proctitis” refers to a disease form where bowel inflammation is limited to the rectum. Because of its limited extent (usually less than the six inches of the rectum), ulcerative proctitis tends to be a milder form of ulcerative colitis. It is associated with fewer complications and offers a better outlook than more widespread disease. For approximately 30% of all patients with ulcerative colitis, the illness begins as ulcerative proctitis.

As used herein, “proctosigmoiditis” refers to a form of colitis affecting the rectum and the sigmoid colon, the lower segment of colon located right above the rectum. Symptoms include bloody diarrhea, cramps, and a constant feeling of the need to pass stool, known as tenesmus. Moderate pain on the lower left side of the abdomen may occur in active disease.

As used herein, “left-sided colitis” refers to continuous inflammation that begins at the rectum and extends as far as a bend in the colon near the spleen called the splenic flexure. Symptoms include loss of appetite, weight loss, diarrhea, severe pain on the left side of the abdomen, and bleeding.

As used herein, “pan-ulcerative (total) colitis” affects the entire colon. Symptoms include diarrhea, severe abdominal pain, cramps, and extensive weight loss. Potentially serious complications include massive bleeding and acute dilation of the colon (toxic megacolon), which may lead to an opening in the bowel wall. Serious complications may require surgery.

Several theories have been proposed for the cause of ulcerative colitis. There is some evidence to suggest that the body's immune system reacts to an environmental, dietary or infectious agent in genetically susceptible individuals causing inflammation in the intestinal wall. The latest postulated causal agent is the to be an infection of the lining with a Fusobacterium varium identified by researchers from Japan. Ulcerative colitis is not caused by emotional distress or sensitivity to certain foods or food products but these factors may trigger symptoms in some people. Ulcerative colitis is most likely not an aberrant reaction but an infection.

The most common symptoms of ulcerative colitis are bloody diarrhea and abdominal pain. Patients also may experience fever, rectal bleeding, fatigue, anaemia, loss of appetite, weight loss and loss of body fluids and nutrients resulting in nutritional deficiencies. These symptoms occur as intermittent attacks in between periods when the symptoms go away (remissions). These disease-free periods can last for months or even years. Usually an attack begins with increased urgency to defecate, mild lower abdominal cramps, and blood and mucus in the stools.

Ulcerative colitis may cause long-term problems such as arthritis, inflammation of the eye, liver disease (fatty liver, hepatitis, cirrhosis, and primary sclerosing cholangitis), osteoporosis, skin rashes, anaemia and kidney stones. These complications may occur when the immune system triggers inflammation in other parts of the body. These problems can disappear when the colitis is treated effectively.

Treatment for ulcerative colitis depends on the seriousness of the disease. Most people are treated with medication. Some people whose symptoms are triggered by certain foods are able to control the symptoms by avoiding foods that upset their intestines, like highly seasoned foods or dairy products. Each person may experience ulcerative colitis differently, so treatment is adjusted for each individual.

Many patients with mild or moderate disease are first treated with 5-ASA agents, including a combination of the drugs 5-aminosalicylic acids and sulfasalazine that helps control inflammation. Sulfasalazine is the most commonly used of these drugs. Sulfasalazine can be used for as long as needed and can be given along with other drugs. Patients who do not do well on sulfasalazine may respond to newer 5-ASA agents. Possible side effects of 5-ASA preparations include nausea, vomiting, heartburn, diarrhea and headache.

People with severe disease and those who do not respond to 5-ASA preparations may be treated with added corticosteroids. Prednisone and budesonide and hydrocortisone are corticosteroids used to reduce inflammation. They can be given orally, intravenously, through an enema, or in a suppository, depending on the location of the inflammation. Corticosteroids can cause side effects such as weight gain, acne, facial hair, hypertension, diabetes, mood swings, and increased risk of infection, so doctors carefully monitor patients taking these medications.

Immunosuppressants such as azathioprine, 6-mercaptopurine (6-MP) and methotrexate are often used and can make a marked improvement at a low dose with few side effects. Other drugs may be given to relax the patient or to relieve pain, diarrhea, or infection.

Occasionally, symptoms are severe enough that the person must be hospitalized. For example, a person may have severe bleeding or severe diarrhea that causes dehydration. In such cases the doctor will try to stop diarrhea and loss of blood, fluids, and mineral salts. The patient may need a special diet, feeding through a vein, medications, or sometimes surgery.

In severe cases, a patient may need surgery to remove the diseased colon. Sometimes the doctor will recommend removing the colon if medical treatment fails or if the side effects of corticosteroids or other drugs threaten the patient's health.

In one aspect, the present disclosure provides a method for reducing the level of calprotectin in a subject in need thereof, where the method comprises treating said patient with a treatment regimen comprising the administration of a pharmaceutical composition comprising live non-pathogenic fecal bacteria for at least 8 weeks and at least three times per week. In another aspect, any method or treatment regimen provided here can also be used to reduce the level of calprotectin and inflammation in a subject in need thereof. In an aspect, the present disclosure provides a method for reducing the level of calprotectin in a subject in need thereof by at least 10% compared to the calprotectin level prior to treatment. In another aspect, the level of calprotectin is reduced by at least 20%, 25%, 30%, 35%, 40%, 45%, 50%, 55%, 60%, 65%, 70%, 75%, 80%, 85%, 90%, or 95%. In another aspect, the level of calprotectin is reduced by between 2 and 10%, 10 and 20%, 20 and 30%, 30 and 40%, 40 and 50%, 50 and 60%, 60 and 70%, 70 and 80%, 80 and 85%, 85 and 90%, 90 and 95%, and 95 and 99% compared to the calprotectin level prior to treatment. In a further aspect, the level of calprotectin is reduced to below 100 μg/g, below 90 μg/g, below 80 μg/g, below 70 μg/g, below 60 μg/g, below 65 μg/g, below 55 μg/g, below 50 μg/g, below 45 μg/g, below 40 μg/g, or below 35 μg/g. In another aspect, the level of calprotectin is reduced in a subject in need thereof following a treatment regimen lasting for at least 8 weeks. In another aspect, the level of calprotectin is reduced in a subject in need thereof at 8 weeks after the completion of the treatment regimen. In yet another aspect, the level of calprotectin is reduced in a subject in need thereof between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks after the completion of the treatment regimen. In a further aspect, the level of calprotectin is reduced in a subject in need thereof between 12 and 30 weeks, between 12 and 28 weeks, between 12 and 20 weeks, between 14 and 20 weeks, between 14 and 26 weeks, between 12 and 18 weeks, between 12 and 16 weeks, between 20 and 30 weeks, between 25 and 30 weeks, and between 21 and 27 weeks after the completion of the treatment regimen. In another aspect, the level of calprotectin is reduced in a subject in need thereof after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 18 or more, 20 or more, 22 or more, 24 or more, 26 or more, 28 or more, 30 or more, 40 or more, 50 or more weeks after the completion of the treatment regimen.

In one aspect, the present disclosure provides a method for reducing the level of lactoferrin in a subject in need thereof, where the method comprises treating said patient with a treatment regimen comprising the administration of a pharmaceutical composition comprising viable non-pathogenic fecal bacteria for at least 8 weeks and at least three times per week. In another aspect, the method comprises treating said patient with a treatment regimen comprising the administration of a pharmaceutical composition comprising viable non-pathogenic fecal bacteria for at least 8 weeks and at least once per week. In another aspect, any method or treatment regimen provided here can also be used to reduce the level of lactoferrin and inflammation in a subject in need thereof. In an aspect, the present disclosure provides a method for reducing the level of lactoferrin in a subject in need thereof by at least 10% compared to the lactoferrin level prior to treatment. In another aspect, the level of lactoferrin is reduced by at least 20%, 25%, 30%, 35%, 40%, 45%, 50%, 55%, 60%, 65%, 70%, 75%, 80%, 85%, 90%, or 95%. In another aspect, the level of lactoferrin is reduced by between 2 and 10%, 10 and 20%, 20 and 30%, 30 and 40%, 40 and 50%, 50 and 60%, 60 and 70%, 70 and 80%, 80 and 85%, 85 and 90%, 90 and 95%, and 95 and 99% compared to the lactoferrin level prior to treatment. In a further aspect, the level of lactoferrin is reduced to below 100 μg/g, below 90 μg/g, below 80 μg/g, below 70 μg/g, below 60 μg/g, below 65 μg/g, below 55 μg/g, below 50 μg/g, below 45 μg/g, below 40 μg/g, below 35 μg/g, below 30 μg/g, below 25 μg/g, below 20 μg/g, below 15 μg/g, or below 10 μg/g. In yet another aspect, the level of lactoferrin is reduced to a normal level of below 7.24 μg/g of feces. In another aspect, the level of lactoferrin is reduced in a subject in need thereof following a treatment regimen lasting for at least 8 weeks. In another aspect, the level of lactoferrin is reduced in a subject in need thereof at 8 weeks after the completion of the treatment regimen. In yet another aspect, the level of lactoferrin is reduced in a subject in need thereof between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks after the completion of the treatment regimen. In a further aspect, the level of lactoferrin is reduced in a subject in need thereof between 12 and 30 weeks, between 12 and 28 weeks, between 12 and 20 weeks, between 14 and 20 weeks, between 14 and 26 weeks, between 12 and 18 weeks, between 12 and 16 weeks, between 20 and 30 weeks, between 25 and 30 weeks, and between 21 and 27 weeks after the completion of the treatment regimen. In another aspect, the level of lactoferrin is reduced in a subject in need thereof after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 18 or more, 20 or more, 22 or more, 24 or more, 26 or more, 28 or more, 30 or more, 40 or more, 50 or more weeks after the completion of the treatment regimen.

In another aspect, this disclosure provides a method of providing a therapeutic dosing regimen to a patient with a gastrointestinal (GI) disorder in need thereof, the method comprising administering to the patient a therapeutic composition comprising fecal microbes based upon a level of a fecal marker for intestinal inflammation, wherein the fecal marker comprises a protein secreted by an immune cell of the patient.

In another aspect, this disclosure provides a method for optimizing the dosing regimen of a fecal microbe-based therapy in a patient with a gastrointestinal disorder in need thereof, the method comprising:
d. administering to the patient a therapeutic composition comprising fecal microbes at a first dosing regimen comprising a first dosage at a first dosing frequency;
e. determining the level of a fecal marker for intestinal inflammation in the patient, wherein the fecal marker comprises a protein secreted by an immune cell of the patient; and
f. modifying the first dosing frequency based on the level of the fecal marker for intestinal inflammation.

In one aspect, a fecal marker for intestinal inflammation comprises one or more peptidases, proteinases, phosphorylating enzyme, glycoprotein, or peroxidase. In another aspect, a fecal marker for intestinal inflammation is secreted or released from one or more secretory cells. In another aspect, a fecal marker for intestinal inflammation is secreted by one or more types of cells selected from the group comprising immune cells, mucosal cells, and epithelial cells.

In one aspect, a fecal marker is calprotectin. In one aspect, a method further comprises increasing a dosage or a dosing frequency of a fecal therapeutic composition by at least 2, 3, 4, 5, 6, 7, 8, 9, or 10 times for one to ten weeks when the patient exhibits a fecal calprotectin level of above 300 μg/g. In one aspect, a method further comprises increasing a dosage or a dosing frequency of a fecal therapeutic composition by at least 3 times for one to ten weeks when the patient exhibits a fecal calprotectin level of above 350, 400, 450, 500, 550, 600, 700, 800, 900, or 1000 μg/g. In one aspect, a method further comprises increasing a dosage or a dosing frequency of a fecal therapeutic composition by at least 1.5, 2, 3, 4, 5, 6, 7, 8, 9, or 10 times for one to ten, two to eight, three to six, or four to five weeks when the patient exhibits a fecal calprotectin level of above 200, 300, 350, 400, 450, 500, 550, 600, 700, 800, 900, or 1000 μg/g. In another aspect, a method further comprises increasing a dosage or a dosing frequency of a fecal therapeutic composition by at least 1.5, 2, 3, 4, 5, 6, 7, 8, 9, or 10 times for one to ten, two to eight, three to six, or four to five weeks when the patient exhibits a fecal calprotectin level of between 50 and 200, 200 and 300 and 350, 350 and 400, 400 and 450, 450 and 500, 500 and 550, 550 and 600, 600 and 700, 700 and 800, 800 and 900, or 900 and 1000 μg/g feces.

In another aspect, a method further comprises gradually decreasing a dosage or a dosing frequency of a fecal therapeutic composition by at least about 20% for at least 2 weeks when the patient exhibits a fecal calprotectin level of below 500 μg/g, and monitoring the fecal calprotectin level in the patient. In one aspect, a dosage or a dosing frequency of a fecal therapeutic composition is decreased by at least about 20%, 30%, 40%, 50%, 60%, or 70% for at least 2 weeks when the patient exhibits a fecal calprotectin level of below 500 μg/g. In one aspect, a dosage or a dosing frequency of a fecal therapeutic composition is decreased by at least about 30% for at least 2, 3, 4, 5, 6, or 7 weeks when the patient exhibits a fecal calprotectin level of below 500 μg/g. In one aspect, a dosage or a dosing frequency of a fecal therapeutic composition is decreased by at least about 30% for at least 4 weeks when the patient exhibits a fecal calprotectin level of below 100, 200, 300, 400, or 500 μg/g. In another aspect, a dosage or a dosing frequency of a fecal therapeutic composition is decreased by at least about 20%, 30%, 40%, 50%, 60%, or 70% for at least 2, 3, 4, 5, 6, 7, or 8 weeks when the patient exhibits a fecal calprotectin level of below 100, 200, 300, 400, or 500 μg/g. In another aspect, a method further comprises maintaining a stable dosing regimen in the patient when a patient exhibits a fecal calprotectin level of below 50 μg/g. In another aspect, a dosage or a dosing frequency of a fecal therapeutic composition is decreased by between 20 and 30%, 30 and 40%, 40 and 50%, 50 and 60%, or 60 and 70% for at least 2, 3, 4, 5, 6, 7, or 8 weeks when the patient exhibits a fecal calprotectin level of between 100 and 200, 200 and 300, 300 and 400, 400 and 500, or 500 and 1000 μg/g feces. In another aspect, a method further comprises maintaining a stable dosing regimen in the patient when a patient exhibits a fecal calprotectin level of between 10 and 20, 20 and 30, 30 and 40, or 40 and 50 μg/g.

In another aspect, a method further comprises gradually decreasing a dosage or a dosing frequency of a fecal therapeutic composition by at least about 20% for at least 2 weeks when the patient's fecal calprotectin level decreases by at least 20% from a baseline level, and monitoring the fecal calprotectin level in the patient. In one aspect, a dosage or a dosing frequency of a fecal therapeutic composition is decreased by at least about 20%, 30%, 40%, 50%, 60%, or 70% for at least 2 weeks when the patient's fecal calprotectin level decreases by at least 20% from a baseline level. In one aspect, a dosage or a dosing frequency of a fecal therapeutic composition is decreased by at least about 30% for at least 2, 3, 4, 5, 6, or 7 weeks when the patient's fecal calprotectin level decreases by at least 20% from a baseline level. In one aspect, a dosage or a dosing frequency of a fecal therapeutic composition is decreased by at least about 30% for at least 4 weeks when the patient's fecal calprotectin level decreases by at least 10%, 20%, 30%, 40%, 50%, or 60% from a baseline level. In another aspect, a dosage or a dosing frequency of a fecal therapeutic composition is decreased by at least about 20%, 30%, 40%, 50%, 60%, or 70% for at least 2, 3, 4, 5, 6, 7, or 8 weeks when the patient's fecal calprotectin level decrease by at least 10%, 20%, 30%, 40%, 50%, or 60% from a baseline level. In a further aspect, a dosage or a dosing frequency of a fecal therapeutic composition is decreased by between 20 and 30%, 30 and 40%, 40 and 50%, 50% and 60, or 60 and 70% for at least 2, 3, 4, 5, 6, 7, or 8 weeks when the patient's fecal calprotectin level is decreased by between 10 and 20%, 20 and 30%, 30 and 40%, 40 and 50%, 50 and 60%, or 60 and 70% from a baseline level.

In one aspect, a fecal marker is lactoferrin. In one aspect, a method further comprises increasing a dosage or a dosing frequency of a fecal therapeutic composition by at least 2, 3, 4, 5, 6, 7, 8, 9, or 10 times for one to ten weeks when the patient exhibits a fecal lactoferrin level of above 7 μg/g. In one aspect, a method further comprises increasing a dosage or a dosing frequency of a fecal therapeutic composition by at least 3 times for one to ten weeks when the patient exhibits a fecal lactoferrin level of above 300, 200, 100, 75, 50, 40, 30, 20, or 10 μg/g. In one aspect, a method further comprises increasing a dosage or a dosing frequency of a fecal therapeutic composition by at least 1.5, 2, 3, 4, 5, 6, 7, 8, 9, or 10 times for one to ten, two to eight, three to six, or four to five weeks when the patient exhibits a fecal lactoferrin level of above 10, 20, 30, 35, 40, 45, 50, 55, 60, 70, 80, 90, or 100 μg/g of feces. In yet another aspect, a method comprises increasing a dosage or a dosing frequency of a fecal therapeutic composition in a patient when a patient exhibits a fecal lactoferrin level of between 10 and 50, 50 and 100, 100 and 150, 150 and 200, 200 and 250, 250 and 300, 300 and 350, or 10 and 400 μg/g of feces.

In another aspect, a method further comprises gradually decreasing a dosage or a dosing frequency of a fecal therapeutic composition by at least about 20% for at least 2 weeks when the patient exhibits a fecal lactoferrin level of below 10, 9, 8, 7, 6, 5, 4, 3, or 2 μg/g of feces, and monitoring the fecal lactoferrin level in the patient. In one aspect, a dosage or a dosing frequency of a fecal therapeutic composition is decreased by at least about 20%, 30%, 40%, 50%, 60%, or 70% for at least 2 weeks when the patient exhibits a fecal lactoferrin level of below 10, 7, 6, 6, 4, 3, or 2 μg/g of feces. In one aspect, a dosage or a dosing frequency of a fecal therapeutic composition is decreased by at least about 30% for at least 2, 3, 4, 5, 6, or 7 weeks when the patient exhibits a fecal lactoferrin level of below 10, 9, 8, 7, 6, 5, 4, 3, or 2 μg/g. In one aspect, a dosage or a dosing frequency of a fecal therapeutic composition is decreased by at least about 30% for at least 4 weeks when the patient exhibits a fecal lactoferrin level of below 10, 9, 8, 7, 6, 5, 4, 3, or 2 μg/g. In another aspect, a dosage or a dosing frequency of a fecal therapeutic composition is decreased by at least about 20%, 30%, 40%, 50%, 60%, or 70% for at least 2, 3, 4, 5, 6, 7, or 8 weeks when the patient exhibits a fecal lactoferrin level of below 10, 9, 8, 7, 6, 5, 4, 3, or 2 μg/g. In another aspect, a method further comprises maintaining a stable dosing regimen in the patient when a patient exhibits a fecal lactoferrin level of below 10, 9, 8, 7, 6, 5, 4, 3, or 2 μg/g. In yet another aspect, a method comprises maintaining a stable dosing regimen in the patient when a patient exhibits a fecal lactoferrin level of between 0 and 7.24, 0 and 7.3, 1 and 7, 2 and 8, 3 and 9, 1 and 10, 0 and 10 or 1 and 8 μg/g.

In another aspect, a method further comprises gradually decreasing a dosage or a dosing frequency of a fecal therapeutic composition by at least about 20% for at least 2 weeks when the patient's fecal lactoferrin level decreases by at least 20% from a baseline level, and monitoring the fecal lactoferrin level in the patient. In one aspect, a dosage or a dosing frequency of a fecal therapeutic composition is decreased by at least about 20%, 30%, 40%, 50%, 60%, or 70% for at least 2 weeks when the patient's fecal lactoferrin level decreases by at least 20% from a baseline level. In one aspect, a dosage or a dosing frequency of a fecal therapeutic composition is decreased by at least about 30% for at least 2, 3, 4, 5, 6, or 7 weeks when the patient's fecal lactoferrin level decreases by at least 20% from a baseline level. In one aspect, a dosage or a dosing frequency of a fecal therapeutic composition is decreased by at least about 30% for at least 4 weeks when the patient's fecal lactoferrin level decreases by at least 10%, 20%, 30%, 40%, 50%, or 60% from a baseline level. In another aspect, a dosage or a dosing frequency of a fecal therapeutic composition is decreased by at least about 20%, 30%, 40%, 50%, 60%, or 70% for at least 2, 3, 4, 5, 6, 7, or 8 weeks when the patient's fecal lactoferrin level decrease by at least 10%, 20%, 30%, 40%, 50%, or 60% from a baseline level. In a further aspect, a dosage or a dosing frequency of a fecal therapeutic composition is decreased by between 20 and 30%, 30 and 40%, 40 and 50%, 50% and 60, or 60 and 70% for at least 2, 3, 4, 5, 6, 7, or 8 weeks when the patient's fecal lactoferrin level is decreased by between 10 and 20%, 20 and 30%, 30 and 40%, 40 and 50%, 50 and 60%, or 60 and 70% from a baseline level.

In one aspect, a fecal marker is both calprotectin and lactoferrin. In one aspect, a method comprises increasing a dosage or a dosing frequency of a fecal therapeutic composition by at least 2, 3, 4, 5, 6, 7, 8, 9, or 10 times for one to ten weeks when the patient exhibits an abnormal fecal calprotectin, an abnormal fecal lactoferrin, or both abnormal fecal calprotectin and lactoferrin levels. In another aspect, the method comprises increasing the dosage or dosing frequency of a fecal therapeutic composition when the fecal calprotectin level is at least 50 μg/g and the lactoferrin level is at least 7.3 μg/g. In yet another aspect, the method comprises decreasing the dosage or dosing frequency of a fecal therapeutic composition when the fecal calprotectin level is at most 50 μg/g and the lactoferrin level is at most 7.3 μg/g. In a further aspect, the method comprises maintaining the dosage or dosing frequency of a fecal therapeutic composition when the fecal calprotectin level is between 0 and 50 μg/g and the lactoferrin level is between 0 and 7.3 μg/g.

In one aspect, this disclosure provides a method for screening a fecal donor, the method comprising administering to a test subject a fecal therapeutic composition derived from the fecal donor, measuring a fecal marker for intestinal inflammation, where the fecal marker comprises a protein secreted by an immune cell in the test subject, and selecting the fecal donor based on the level of the fecal marker for intestinal inflammation.

In a further aspect, this disclosure provides a method for determining potency of a pharmaceutical composition (e.g., donor-derived lyophilised microbe or microbiota composition) in treating a GI disorder, the method comprising administering the pharmaceutical composition to a test subject with a GI disorder and monitoring the test subject's response to the pharmaceutical composition by measuring the level of a fecal marker for intestinal inflammation. In one aspect, such a method can be used to select a suitable or preferred donor.

In a further aspect, a method provided herein is for treating, providing a therapeutic dosing regimen to a patient with, optimizing the dosing regimen of a fecal microbe-based therapy for, or selecting a fecal donor for a condition (including GI disorder or a condition having a GI component) or for improving the efficacy of a fecal microbiota-based therapy for a condition, where the condition is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, and a mood disorder.

In a further aspect, a method provided herein is for treating, providing a therapeutic dosing regimen to a patient with, optimizing the dosing regimen of a fecal microbe-based therapy for, or selecting a fecal donor for Antibiotic Associated Colitis, Chronic Clostridium difficile Infection (CDI), Chronic constipation, Chronic Fatigue Syndrome (CFS), Collagenous Colitis, Colonic Polyps, Constipation Predominant FBD, Crohn's Disease, Functional Bowel Disease (FBD), Gastro-oesophageal Reflux, Irritable bowel syndrome (IBS) constipation-predominant, IBS diarrhea/constipation alternating, IBS diarrhea-predominant, IBS pain-predominant, Indeterminate Colitis, Inflammatory Bowel Disease (IBD), Microscopic Colitis, Mucous Colitis, Multiple Sclerosis, Non-ulcer Dyspepsia, Norwalk Viral Gastroenteritis, Pain Predominant FBD, Primary Clostridium difficile Infection (CDI), Primary Sclerosing Cholangitis (PSC), Pseudomembranous Colitis, Small Bowel Bacterial Overgrowth, Ulcerative Colitis, and Upper Abdominal FBD.

In one aspect, a fecal marker for intestinal inflammation comprises a protein secreted by a secretory cell. In another aspect, the protein is selected from the group consisting of calprotectin, lactoferrin, M2-pyruvate kinase (M2-PK), neopterin, metalloproteinases, myeloperoxidases, and polymorphonuclear elastase. One or more these fecal markers can be tested together or in tandem. Exemplary assays for testing these markers can be found, for example, in Lehmann et al., Therap Adv Gastroenterol. 2015 January; 8(1): 23-36; Assche, Gastroenterol Hepatol (N Y). 2011 June; 7(6): 396-398; and Judd et al., J Gastroenterol Hepatol. 2011 October; 26(10):1493-9. In another aspect, another noninvasive marker (either fecal or non-fecal) can be used in lieu of a fecal marker for intestinal inflammation. Such noninvasive markers include, e.g., serological biomarkers (e.g., erythrocyte sedimentation rate (ESR), white blood cell count and C-reactive protein (CRP)), rectal nitric oxide, fecal Eosinophil protein X (EPX). See Turkey and Kasapoglu, Clinics, vol. 65 no. 2 (2010).

In one aspect, the present disclosure provides a method for treating ulcerative colitis in a subject in need thereof, where the method comprises treating said patient with a treatment regimen comprising the administration of a pharmaceutical composition comprising live non-pathogenic fecal bacteria for at least 8 weeks and at least three times per week. In another aspect, any method or treatment regimen provided here can also be used to treat one or more indications selected from the group consisting of collagenous colitis, lymphocytic colitis, Crohn's colitis, diverticulitis, and pouchitis.

In an aspect, this disclosure provides a method for treating ulcerative colitis in a subject in need thereof and exhibiting a Mayo endoscopy score of 3 or lower, where the method comprises administering to said subject a pharmaceutical composition comprising live non-pathogenic fecal bacteria. In one aspect, this administering is following a treatment regimen lasting for at least 8 weeks. In an aspect, this administering is following a treatment regimen of at least 8 weeks and at least three time per week. In one aspect, this administering is following a treatment regimen of at least 8 weeks and at least three times per week.

In an aspect, the present disclosure provides a method for treating ulcerative colitis in a subject in need thereof, where the method comprises administering to the subject a pharmaceutical composition comprising live non-pathogenic fecal bacteria, where the subject has no concomitant corticosteroid use during the method and has no corticosteroid use immediately prior to commencing the method. In one aspect, this administering is following a regimen lasting for at least 9 weeks. In an aspect, this administering is following a regimen of at least 8 weeks and at least three times per week.

In an aspect, the subject of the present disclosure exhibits a Mayo score of at least 4 prior to treatment, such as a Mayo score of 4, 5, 6, 7, 8, 9, 10. In one aspect, the subject of the present disclosure exhibits a Mayo score of 4 to 10 prior to treatment, such as 4 to 9, 5 to 10, 5 to 8, or 6 to 8.

In an aspect, the subject of the present disclosure exhibits an UCEIS score of at least 4 prior to treatment, such as an UCEIS score of 4, 5, 6, 7, 8, 9, 10. In one aspect, the subject of the present disclosure exhibits a UCEIS score of 4 to 10 prior to treatment, such as 4 to 9, 5 to 10, 5 to 8, or 6 to 8.

In one aspect, the subject of the present disclosure is capable of achieving a primary outcome at the end of a treatment regimen, where the primary outcome is defined as a steroid-free clinical remission and endoscopic remission or response at the end of the treatment regimen, where the steroid free clinical remission is defined as a total Mayo score of 2 or lower with sub-scores of 1 or lower, and where the endoscopic remission or response is defined as a reduction of at least 1 point from baseline in endoscopy score. In another aspect, the subject of the present disclosure is capable of achieving a primary outcome at the end of a treatment regimen, where the primary outcome is defined as a steroid-free clinical remission which is defined as a total Mayo score of 2 or lower with sub-scores of 1 or lower. In a further aspect, the subject of the present disclosure is capable of achieving a primary outcome at the end of a treatment regimen, where the primary outcome is defined as a steroid-free endoscopic remission or response which is defined as a reduction of at least 1 point from baseline in endoscopy score.

In an aspect, a subject of the present disclosure has no steroid use within at least one week prior to commencing the methods provided herein. In another aspect, a subject of the present disclosure has no steroid use within at least two, three, four, or five weeks prior to commencing the methods provided herein. In a further aspect, a subject of the present disclosure has no steroid use within at least 1, 2, 3, 4, 5, 6, 7, 8, 9, 10, 11, 12, 13, or 14 days prior to commencing the methods provided herein. In one aspect, a steroid may be prednisone, budesonide, or hydrocortisone. In an aspect, a subject of the present disclosure has no corticosteroid use within at least one week prior to commencing the methods provided herein. In one aspect, a subject of the present disclosure has no corticosteroid use prior to commencing the methods provided herein.

In some aspects, the methods of the present disclosure further comprise determining the subject's baseline gut bacterial diversity. In an aspect, a subject's baseline gut bacterial diversity is assessed by analyzing Shannon's diversity of the subject's fecal sample prior to the treating step. In one aspect, a subject's baseline fecal Shannon diversity is between 0.5 and 2.2 based on bacterial species level, such as between 0.5 and 2.0, between 1.0 and 2.2, or between 1.0 and 1.5. In an aspect, a subject's fecal Shannon diversity increases by at least 50%, 60%, 70%, 80%, 90%, 95%, 98%, 99%, 99.5%, 99.8%, or 99.9% compared to before treatment. In one aspect, a subject's fecal Shannon diversity increases by at least 1, 2, 3, 4, 5, 6, 7, 8, 9, 10, 11, 15, 20, or 30 folds compared to before treatment. In one aspect, a subject's post-treatment fecal Shannon diversity is between 1.5 and 6.0 based on bacterial species level, such as between 1.5 and 5.0, between 1.5 and 4.5, between 1.5 and 4.0, between 1.5 and 3.5, between 1.5 and 3.0, between 1.5 and 2.5, between 1.5 and 2.0, between 2.0 and 4.5, between 2.5 and 4.0, between 3.0 and 3.5, between 2.0 and 6.0, between 2.5 and 6.0, between 3.0 and 6.0, between 3.5 and 6.0, between 4.0 and 6.0, between 4.5 and 6.0, between 5.0 and 6.0, and between 5.5 and 6.0.

In certain aspects, the methods of the present disclosure further comprise determining the level of Fusobacterium, Sutterella, or both in a subject's gut. In some aspects, methods of the present disclosure further comprise determining the level of one or more bacteria selected from the group consisting of Barnesiella, Parabacteroides, Clostridium IV, Ruminococcus, Blautia, Dorea, Ruminococcus2, and Clostridium XVIII in the subject's gut.

In an aspect, the present disclosure provides a treatment regimen that is capable of achieving a primary outcome rate of at least two fold higher relative to a primary outcome rate from placebo, where the primary outcome is defined as a steroid-free clinical remission and endoscopic remission or response at the end of the treatment regimen, where the clinical remission is defined as a total Mayo score of 2 or lower with all sub-scores of 1 or lower, and where the endoscopic remission or response is defined as a reduction of at least 1 point from baseline in Mayo endoscopy score. In one aspect, the present disclosure provides a treatment regimen that is capable of achieving a primary outcome rate higher than a primary outcome rate from placebo, where the primary outcome is defined as a steroid-free clinical remission and endoscopic remission or response at the end of the treatment regimen, where the clinical remission is defined as a total Mayo score of 2 or lower with all sub-scores of 1 or lower, and where the endoscopic remission or response is defined as a reduction of at least 1 point from baseline in Mayo endoscopy score.

In one aspect, a treatment regimen in accordance with the present disclosure is capable of achieving a primary outcome rate of at least 25%, such as at least 20%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, at least 99.5%, or at least 99.9%. In an aspect, a treatment regimen is capable of achieving a primary outcome rate of between 20% to 40%, such as between 20% and 35%, between 25% and 40%, between 25% and 35%, between 25% and 30%, or between 30% and 35%.

In one aspect, a treatment regimen in accordance with the present disclosure is capable of achieving a clinical remission sustaining rate of at least 40% at 8 weeks after the completion of the treatment regimen. In an aspect, a treatment regimen is capable of achieving a clinical remission sustaining rate of at least 45%, such as at least 50%, at least 55%, at least 60%, at least 65%, at least 70%, at least 75%, at least 80%, at least 85%, at least 90%, at least 95%, at least 98%, at least 99%, at least 99.5%, or at least 99.9% at 8 weeks after the completion of the treatment regimen. In an aspect, a treatment regimen is capable of achieving a clinical remission sustaining rate of between 35% and 60%, such as between 35% and 55%, between 40% and 60%, between 40% and 55%, between 40% and 50%, between 45% and 55%, or between 45% and 50% at 8 weeks after the completion of the treatment regimen.

In one aspect, a treatment regimen in accordance with the present disclosure is capable of achieving a steroid-free clinical remission rate of at least two fold higher relative to a steroid-free clinical remission rate from placebo, where the clinical remission is defined as a combined Mayo score of 1 or lower for rectal bleeding and stool frequency. In an aspect, a treatment regimen in accordance with the present disclosure is capable of achieving a steroid-free clinical remission rate higher than a steroid-free clinical remission rate from placebo, where the clinical remission is defined as a combined Mayo score of 1 or lower for rectal bleeding and stool frequency. In an aspect, a treatment regimen is capable of achieving a steroid-free clinical remission rate of at least 40%, such as at least 45%, at least 50%, at least 55%, at least 60%, at least 65%, at least 70%, at least 75%, at least 80%, at least 85%, at least 90%, at least 95%, at least 98%, at least 99%, at least 99.5%, or at least 99.9%. In one aspect, a treatment regimen is capable of achieving a steroid-free clinical remission rate of between 35% and 55%, such as between 40% and 55%, between 35% and 50%, between 40% and 50%, between 40% and 45%, or between 45% and 50%.

In an aspect, a treatment regimen in accordance with the present disclosure is capable of achieving a steroid-free clinical response rate of at least two fold higher relative to a steroid-free clinical response rate from placebo, where the clinical response is defined as a total Mayo score decrease of 3 or higher or a 50% higher reduction from baseline in combined score for rectal bleeding and stool frequency. In one aspect, a treatment regimen in accordance with the present disclosure is capable of achieving a steroid-free clinical response rate higher than a steroid-free clinical response rate from placebo, where the clinical response is defined as a total Mayo score decrease of 3 or higher or a 50% higher reduction from baseline in combined score for rectal bleeding and stool frequency. In one aspect, a treatment regimen is capable of achieving a steroid-free clinical response rate of at least 50%, such as at least 55%, at least 60%, at least 65%, at least 70%, at least 75%, at least 80%, at least 85%, at least 90%, at least 95%, at least 98%, at least 99%, at least 99.5%, or at least 99.9%. In an aspect, a treatment regimen in accordance with the present disclosure is capable of achieving a steroid-free clinical response rate between 45% and 65%, such as between 45% and 60%, between 50% and 65%, between 50% and 60%, between 50% and 55%, or between 55% and 60%.

In one aspect, a treatment regimen in accordance with the present disclosure is capable of achieving an endoscopic response rate of at least two fold higher relative to an endoscopic response rate from placebo, where the endoscopic response is defined as a total UCEIS score decrease of 3 or higher or a 50% or higher reduction from baseline. In one aspect, a treatment regimen in accordance with the present disclosure is capable of achieving an endoscopic response rate higher than an endoscopic response rate from placebo, where the endoscopic response is defined as a total UCEIS score decrease of 3 or higher or a 50% or higher reduction from baseline. In an aspect, a treatment regimen is capable of achieving an endoscopic rate of at least 30%, such as at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, at least 99.5%, or at least 99.9%. In one aspect, a treatment regimen is capable of achieving an endoscopic response rate between 30% and 45%, such as between 30% and 40%, between 35% and 45%, or between 35% and 40%.

In one aspect, the present disclosure provides a method for treating ulcerative colitis in a subject in need thereof, where the method comprises administering to the subject a pharmaceutically active dose of a therapeutic composition comprising live non-pathogenic fecal bacteria. In another aspect, this disclosure provides use of a composition comprising live non-pathogenic fecal bacteria in the manufacture of a medication for the treatment of ulcerative colitis.

In some aspects, methods of the present disclosure treat a form of ulcerative colitis selected from the group consisting of ulcerative proctitis, proctosigmoiditis, left-sided colitis, and pan-ulcerative colitis. In an aspect, a pharmaceutical composition in accordance with the present disclosure comprises a fecal microbiota preparation. In one aspect, a pharmaceutical composition comprises an isolated or purified population of live non-pathogenic fecal bacteria. In an aspect, a pharmaceutical composition comprises a non-selective fecal microbiota. In one aspect, a pharmaceutical composition comprises a non-selected and substantially complete fecal microbiota. In an aspect, a pharmaceutical composition comprises a full-spectrum fecal microbiota. In one aspect, a method further comprises administering a 5-aminosalicylic acid agent, a corticosteroid, an immunosuppressant, or a combination thereof. In another aspect, a method further comprises administering 5-aminosalicylic acid or a derivative thereof, sulfasalazine or a derivative thereof, or a combination thereof.

In one aspect, the present disclosure provides a method for selecting a treatment plan for treating ulcerative colitis in a subject in need thereof, where the method comprises determining the level of Fusobacterium, Sutterella, or both in the subject's gut; and recommending a fecal bacteria-based therapy when the level of Fusobacterium, Sutterella, or both is above a predetermined level. In an aspect, the level of Fusobacterium, Sutterella, or both is about 8% above a predetermined level, such as about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, or about 200% above a predetermined level. In an aspect, the present disclosure provides a method for selecting a treatment plan for treating ulcerative colitis in a subject in need thereof, where the method comprises determining the level of Fusobacterium, Sutterella, or both in said subject's gut; and recommending a fecal bacteria-based therapy when said level of Fusobacterium, Sutterella, or both is between a predetermined range. In one aspect, the predetermined range is about 8% to about 50% above a predetermined level, such as about 8% to about 40%, about 10% to 50%, about 15% to about 40%, about 20% to about 35%, or about 25% to about 30% above a predetermined level. In an aspect, the predetermined range is about 50% to about 200% above a predetermined level, such as about 50% to about 150%, about 50% to about 100%, about 100% to 150%, about 80% to about 120%, about 90% to about 110%, or about 98% to about 100% above a predetermined level. In some aspects, the level of one or more bacteria is determined via analyzing a subject's feces.

In an aspect, the present disclosure provides a method for selecting a treatment plan for treating ulcerative colitis in a subject in need thereof, where the method comprises determining the level of one or more bacteria selected from the group consisting of Barnesiella, Parabacteroides, Clostridium IV, Ruminococcus, Blautia, Dorea, Ruminococcus2, and Clostridium XVIII in said subject's gut; and recommending a fecal bacteria-based therapy when the level of the one or more selected bacteria is above a predetermined level. In an aspect, the level of the one or more selected bacteria is about 8% above a predetermined level, such as about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, or about 200% above a predetermined level. In an aspect, the present disclosure provides a method for selecting a treatment plan for treating ulcerative colitis in a subject in need thereof, where the method comprises determining the level of one or more bacteria selected from the group consisting of Barnesiella, Parabacteroides, Clostridium IV, Ruminococcus, Blautia, Dorea, Ruminococcus2, and Clostridium XVIII in said subject's gut; and recommending a fecal bacteria-based therapy when the level of the one or more selected bacteria is between a predetermined range. In one aspect, the predetermined range is about 8% to about 50% above a predetermined level, such as about 8% to about 40%, about 10% to 50%, about 15% to about 40%, about 20% to about 35%, or about 25% to about 30% above a predetermined level. In an aspect, the predetermined range is about 50% to about 200% above a predetermined level, such as about 50% to about 150%, about 50% to about 100%, about 100% to 150%, about 80% to about 120%, about 90% to about 110%, or about 98% to about 100% above a predetermined level. In some aspects, the level of one or more bacteria is determined via analyzing a subject's feces.

In one aspect, a predetermined level is established by the corresponding level of the one or more selected bacteria in healthy subjects. In an aspect, a predetermined level is established by the corresponding level of the one or more selected bacteria in healthy subjects in the same demographic category as the subject. In one aspect, a predetermined level is established by the abundance of the total Clostridium or Bacteriodetes population in the same subject.

In one aspect, the present disclosure provides a method which eliminates or reduces one or more ulcerative colitis symptoms selected from the group consisting of diarrhea, cramp, tenesmus, weight loss, bleeding, loss of appetite, abdominal pain, fever, fatigue, anaemia, inflammation, and micro-ulcers.

In one aspect, the present disclosure provides a method for treating ulcerative colitis in a subject in need thereof, where the method comprises administering to the subject a pharmaceutically active dose of a therapeutic composition comprising live non-pathogenic bacteria. In one aspect, the present disclosure provides a method for treating ulcerative colitis in a subject in need thereof, where the method comprises administering daily to the subject a pharmaceutically active dose of a therapeutic composition comprising live non-pathogenic fecal bacteria. In one aspect, a therapeutic composition is administered to an ulcerative colitis patient in need thereof at least once daily for at least two consecutive days. In one aspect, a therapeutic composition is administered at least once daily for at least 3, 4, 5, 6, 7, 8, 9, 10, 11, 12, 13, 14, or 15 consecutive days. In another aspect, a therapeutic composition is administered at least once daily for at least 1, 2, 3, 4, 5, 6, 7, 8, 9, 10, 11, or 12 consecutive weeks. In one aspect, a therapeutic composition is administered at least once daily for at most 4, 5, 6, 7, 8, 9, 10, 11, 12, 13, 14, 15, 16, 17, 18, 19, or 20 consecutive days or weeks. In another aspect, a therapeutic composition is administered at least once daily for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an ulcerative colitis patient in need thereof at least twice daily for at least two consecutive days. In one aspect, a therapeutic composition is administered at least twice daily for at least 3, 4, 5, 6, 7, 8, 9, 10, 11, 12, 13, 14, or 15 consecutive days. In another aspect, a therapeutic composition is administered at least twice daily for at least 1, 2, 3, 4, 5, 6, 7, 8, 9, 10, 11, or 12 consecutive weeks. In one aspect, a therapeutic composition is administered at least twice daily for at most 4, 5, 6, 7, 8, 9, 10, 11, 12, 13, 14, 15, 16, 17, 18, 19, or 20 consecutive days or week. In another aspect, a therapeutic composition is administered at least twice daily for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an ulcerative colitis patient in need thereof at least three times daily for at least two consecutive days. In one aspect, a therapeutic composition is administered at least three times daily for at least 3, 4, 5, 6, 7, 8, 9, 10, 11, 12, 13, 14, or 15 consecutive days. In another aspect, a therapeutic composition is administered at least three times daily for at least 1, 2, 3, 4, 5, 6, 7, 8, 9, 10, 11, or 12 consecutive weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating ulcerative colitis in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating ulcerative colitis in a subject in need thereof, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, the present disclosure provides a method for treating a subject in need thereof, where the method comprises administering to the subject a pharmaceutically active dose of a therapeutic composition comprising fecal microbiota of multiple carefully screened, healthy donors. In an aspect, a subject is administered a therapeutic composition over a dosing period wherein a first dose comprises at least one therapeutic composition comprising fecal microbiota of a single donor, and a second dose of a therapeutic composition comprising fecal microbiota of a single donor different from the donor of the first dose. In another aspect, a first dose comprises a therapeutic composition comprising fecal microbiota of a single donor and a second dose comprises fecal microbiota of a donor pool. The first and second dose do not indicate the order of administration to a subject, but rather that fecal microbiota from separate donors may be used in a non-blended form. In yet another aspect, the fecal microbiota from multiple carefully screened, healthy donors is provided in a blended form.

In an aspect, the present disclosure provides for methods for treating a subject in need thereof by administering to the subject a pharmaceutically active dose of a therapeutic composition comprising fecal microbiota of a single donor. In another aspect, the administering is followed by testing to determine the efficacy of the pharmaceutically active dose of the therapeutic composition. In another aspect, the testing of the subject provides results to determine if the active dose of the therapeutic composition should be adjusted. In another aspect, the testing is followed by administration of a therapeutic composition comprising blended fecal microbiota from multiple donors. In one aspect of the present disclosure, methods provide for treating a subject in need thereof comprising: (1) administering to the subject a first pharmaceutically active dose of a therapeutic composition comprising fecal microbiota of a single donor; (2) testing of the subject to determine efficacy, if an additional dose is necessary, or if the dose should be adjusted; (3) administration of a second therapeutic composition comprising blended fecal microbiota from multiple donors; (4) optionally testing of the subject to determine efficacy, if an additional dose is necessary, or if the dose should be adjusted; and (5) optionally administration of a third therapeutic composition comprising blended fecal microbiota from multiple donors, where the multiple donors (a) comprise all donors from the second therapeutic composition and additional donors, (b) comprise donors not included in the second therapeutic composition, (c) comprise some but not all of the donors from the second therapeutic composition, or comprise donors not included in the second therapeutic composition. In another aspect, the first, second, and third therapeutic compositions are administered in a different order (i.e., first, third, second; third, second, first; third, first, second; second, first, third, etc.).

In another aspect, the present disclosure provides for methods for treating a subject in need thereof with capsules containing a therapeutic composition comprising fecal microbiota from a single donor. In another aspect, a capsule comprises a therapeutic composition comprising fecal microbiota from multiple donors. In one aspect, a subject is administered two or more pills comprising fecal microbiota from a single but different donor.

In one aspect, the present disclosure provides for methods for treating a subject in need thereof comprising administering a therapeutic composition orally or by infusions through a colonoscope, an enema or via a nasogastric or nasojejunal tube. In another aspect, each administration comprises a therapeutic composition comprising fecal microbiota of a single donor similar to or different from a prior administration in a treatment period. In another aspect, a treatment period includes administration of a first dost comprising a therapeutic composition comprising fecal microbiota of a single donor and administration of a second dose comprising a therapeutic composition comprising fecal microbiota of multiple donors.

In one aspect, a subject being treated is a subject already with ulcerative colitis. Administration of a disclosed therapeutic composition to clinically, asymptomatic human subject who is genetically predisposed or prone to ulcerative colitis is also useful in preventing the onset of clinical symptoms of ulcerative colitis. A human subject genetically predisposed or prone to ulcerative colitis can be a human subject having a close family member or relative exhibiting or having suffered ulcerative colitis. In another aspect, a subject being treated is a subject in which ulcerative colitis is to be prevented. In another aspect, a subject being treated is predisposed or susceptible to ulcerative colitis. In another aspect, a subject being treated is a subject diagnosed as having ulcerative colitis. In one aspect, a subject being treated is a patient in need thereof.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old old patient (>85 years).

In one aspect, a method comprises administering a therapeutic composition orally, by enema, or via rectal suppository. In one aspect, a therapeutic composition administered herein is formulated as an enteric coated (and/or acid-resistant) capsule or microcapsule, or formulated as part of or administered together with a food, a food additive, a dairy-based product, a soy-based product or a derivative thereof, a jelly, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In a further aspect, a method further comprises pretreating a subject with an anti-inflammatory drug prior to administration of a therapeutic bacterial or microbiota composition.

In one aspect, a method achieves a remission, cure, response, or resolution rate of ulcerative colitis of at least about 10%, 15%, 20%, 25%, 30%, 35%, 40%, 45%, 50%, 55%, 60%, 65%, 70%, 75%, 80%, 85%, 90%, 95%, 97%, or 99%. In one aspect, a treatment method achieves a reduction of ulcerative colitis disease activity index (UCDAI) after 8 weeks of treatment by more than 2, 3, 4, 5, 6, 7, 8, 9, 10, or 11. In another aspect, a treatment method achieves a reduction of ulcerative colitis disease activity index (UCDAI) after 8 weeks of treatment by more than 2, 3, 4, 5, 6, 7, 8, 9, 10, or 11 in at least 10%, 20%, 30%, 50%, 60%, 70%, 80%, or 90% patients in a patient population. In one aspect, a treatment method achieves at least 10%, 20%, 30%, 50%, 60%, 70%, 80%, or 90% reduction of ulcerative colitis disease activity index (UCDAI) after 8 weeks of treatment compared to baseline (e.g., immediately prior to treatment). In one aspect, a treatment method achieves at least 10%, 20%, 30%, 50%, 60%, 70%, 80%, or 90% reduction of ulcerative colitis disease activity index (UCDAI) in at least 10%, 20%, 30%, 50%, 60%, 70%, 80%, or 90% patients after 8 weeks of treatment compared to baseline (e.g., immediately prior to treatment).

In a further aspect, a patient is assessed using the Disease Activity Index (DAI) or Mayo score system as described in Schroeder et al., Coated oral 5-aminosalcylic acid therapy for mildly to moderately active ulcerative colitis. N Eng J Med. 1987; 317:1625-1629. In one aspect, a treatment method achieves at least 10%, 20%, 30%, 50%, 60%, 70%, 80%, or 90% reduction of Mayo score after 8 weeks of treatment compared to baseline (e.g., immediately prior to treatment). In one aspect, a treatment method achieves at least 10%, 20%, 30%, 50%, 60%, 70%, 80%, or 90% reduction of Mayo score in at least 10%, 20%, 30%, 50%, 60%, 70%, 80%, or 90% patients after 8 weeks of treatment compared to baseline (e.g., immediately prior to treatment).

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cfu. In another aspect, a pharmaceutically active therapeutic effective dose comprises at most about 10⁵, 10⁶, 10⁷, 10⁸, 10⁹, 10¹⁰, 10¹¹, 10¹², or 10¹³ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, 10¹⁰ from 10⁹ cfu to 10¹¹ cfu, from 10⁹ cfu to cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu.

In one aspect, a pharmaceutically active or therapeutic effective dose comprises at least about 10⁵, 10⁶, 10⁷, 10⁸, 10⁹, 10¹⁰, 10¹¹, 10¹² or 10¹³ cells or spores. In another aspect, a pharmaceutically active or therapeutic effective dose comprises at most about 10⁵, 10⁶, 10⁷, 10⁸, 10⁹, 10¹⁰, 10¹¹, 10¹², or 10¹³ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia. In one aspect, a therapeutic composition administered herein comprises one or more, two or more, three or more, four or more, or five or more isolated, purified, or cultured microorganisms selected from the group consisting of Acidaminococcus, Acinetobacter, Akkermansia, Alistipes, Anaerotruncus, Bacteroides, Bifidobacterium Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, or a combination thereof.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In another aspect, a therapeutic composition administered herein comprises a fecal microbiota further supplemented, spiked, or enhanced with a fecal microorganism. In one aspect, a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a therapeutic composition administered herein comprises a fecal microbiota further supplemented, spiked, or enhanced with a species of Veillonellaceae, Firmicutes, Gammaproteobacteria, Bacteroidetes, or a combination thereof. In another aspect, a therapeutic composition administered herein comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In a further aspect, a therapeutic composition comprises fecal bacteria from multiple donors. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a fecal microbiota in a therapeutic composition comprises a donor's substantially entire or non-selective fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of ulcerative colitis. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium sub terminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹², or 10¹³ cfu. In another aspect, a therapeutic composition comprises at most about 10⁵, 10⁶, 10⁷, 10⁸, 10⁹, 10¹⁰, 10¹¹, 10¹², 10¹³ or 10¹⁴ cfu.

In another aspect, a therapeutic composition comprises at least about 10⁵, 10⁶, 10⁷, 10⁸, 10⁹, 10¹⁰, 10¹¹, 10¹², or 10¹³ cells. In another aspect, a therapeutic composition comprises at most about 10⁵, 10⁶, 10⁷, 10⁸, 10⁹, 10¹⁰, 10¹¹, 10¹², 10¹³ or 10¹⁴ cells.

From the foregoing, it will be appreciated that the present invention can be embodied in various ways, which include but not limited to the following embodiments:

Embodiment 1. A method for treating ulcerative colitis (UC) in a subject in need thereof comprising treating the subject with a treatment regimen comprising the administration of a pharmaceutical composition comprising live non-pathogenic fecal bacteria for at least 8 weeks and at least three times per week.

Embodiment 2. A method for treating a condition in a subject in need thereof comprising treating the subject with a treatment regimen comprising the administration of a pharmaceutical composition comprising live non-pathogenic fecal bacteria for at least 8 weeks and at least three times per week, where the condition is selected from the group consisting of collagenous colitis, lymphocytic colitis, Crohn's colitis, diverticulitis, and pouchitis.

Embodiment 3. The method of embodiment 1 or 2, where the treatment regimen is capable of achieving a primary outcome rate of at least two fold higher relative to a primary outcome rate from placebo, where the primary outcome is defined as a steroid-free clinical remission and endoscopic remission or response at the end of the treatment regimen, where the clinical remission is defined as a total Mayo score of 2 or lower with all sub-scores of 1 or lower, where the endoscopic remission or response is defined as a reduction of at least 1 point from baseline in Mayo endoscopy score.

Embodiment 4. The method of embodiment 3, where the treatment regimen is capable of achieving a primary outcome rate of at least 25%.

Embodiment 5. The method of embodiment 3, where the treatment regimen is capable of achieving a primary outcome rate between 20% and 40%.

Embodiment 6. The method of embodiment 3, where the treatment regimen is capable of achieving a clinical remission sustaining rate of at least 40% at 8 weeks after the completion of the treatment regimen.

Embodiment 7. The method of embodiment 3, where the treatment regimen is capable of achieving a clinical remission sustaining rate of between 35% and 60% at 8 weeks after the completion of the treatment regimen.

Embodiment 8. The method of embodiment 1 or 2, where the treatment regimen is capable of achieving a steroid-free clinical remission rate of at least two fold higher relative to a steroid-free clinical remission rate from placebo, where the clinical remission is defined as a combined Mayo score of 1 or lower for rectal bleeding and stool frequency.

Embodiment 9. The method of embodiment 8, where the treatment regimen is capable of achieving a steroid-free clinical remission rate of at least 40%.

Embodiment 10. The method of embodiment 8, where the treatment regimen is capable of achieving a steroid-free clinical remission rate between 35% and 55%.

Embodiment 11. The method of embodiment 1 or 2, where the treatment regimen is capable of achieving a steroid-free clinical response rate of at least two fold higher relative to a steroid-free clinical response rate from placebo, where the clinical response is defined as a total Mayo score decrease of 3 or higher or a 50% or higher reduction from baseline in combined score for rectal bleeding and stool frequency.

Embodiment 12. The method of embodiment 11, where the treatment regimen is capable of achieving a steroid-free clinical response rate of at least 50%.

Embodiment 13. The method of embodiment 11, where the treatment regimen is capable of achieving a steroid-free clinical response rate between 45% and 65%.

Embodiment 14. The method of embodiment 1 or 2, where the treatment regimen is capable of achieving an endoscopic response rate of at least two fold higher relative to an endoscopic response rate from placebo, where the endoscopic response is defined as a total UCEIS score decrease of 3 or higher or a 50% or higher reduction from baseline.

Embodiment 15. The method of embodiment 14, where the treatment regimen is capable of achieving an endoscopic response rate of at least 30%.

Embodiment 16. The method of embodiment 14, where the treatment regimen is capable of achieving an endoscopic response rate between 30% and 45%.

Embodiment 17. The method of embodiment 1 or 2, where the method further comprises determining the subject's baseline gut bacterial diversity.

Embodiment 18. The method of embodiment 17, where the subject's baseline gut bacterial diversity is assessed by analyzing Shannon's diversity of the subject's fecal sample prior to the treating step.

Embodiment 19. The method of embodiment 18, where the subject's fecal Shannon's diversity is between 0.5 and 2.2 based on bacterial species level.

Embodiment 20. The method of embodiment 1 or 2, where the method further comprises determining the level of Fusobacterium, Sutterella, or both in the subject's gut.

Embodiment 21. The method of embodiment 1 or 2, where the method further comprises determining the level of one or more bacteria selected from the group consisting of Barnesiella, Parabacteroides, Clostridium IV, Ruminococcus, Blautia, Dorea, Ruminococcus2, and Clostridium XVIII in the subject's gut.

Embodiment 22. The method of embodiment 1 or 2, where the pharmaceutical composition comprises a fecal microbiota preparation.

Embodiment 23. The method of embodiment 1 or 2, where the subject exhibits a Mayo score of at least 4 prior to the treating step.

Embodiment 24. The method of embodiment 1 or 2, where the subject exhibits a Mayo score of 4 to 10 prior to the treating step.

Embodiment 25. A method for treating ulcerative colitis (UC) in a subject in need thereof and exhibiting a Mayo endoscopy score of 3 or lower, the method comprising administering to the subject a pharmaceutical composition comprising live non-pathogenic fecal bacteria.

Embodiment 26. The method of embodiment 25, where the administering is following a treatment regimen lasting for at least 8 weeks.

Embodiment 27. The method of embodiment 25, where the administering is following a treatment regimen of at least 8 weeks and at least three times per week.

Embodiment 28. The method of embodiment 27, where the subject is capable of achieving a primary outcome at the end of the treatment regimen, where the primary outcome is defined as a steroid-free clinical remission and endoscopic remission or response at the end of the treatment regimen, where the steroid-free clinical remission is defined as a total Mayo score of 2 or lower with all sub-scores of 1 or lower, where the endoscopic remission or response is defined as a reduction of at least 1 point from baseline in endoscopy score.

Embodiment 29. The method of embodiment 25, where the administering step is following a treatment regimen of daily for at least 8 weeks.

Embodiment 30. A method for treating ulcerative colitis (UC) in a subject in need thereof, the method comprising administering to the subject a pharmaceutical composition comprising live non-pathogenic fecal bacteria, where the subject has no concomitant corticosteroid use during said method and has no corticosteroid use immediately prior to commencing the method.

Embodiment 31. The method of embodiment 30, where the subject has no steroid use within at least one week prior to commencing the method.

Embodiment 32. The method of embodiment 30, where the subject has no corticosteroid use within at least one week prior to commencing the method.

Embodiment 33. The method of embodiment 30, where the subject has no corticosteroid use prior to commencing the method.

Embodiment 34. The method of embodiment 30, where the administering is following a regimen lasting for at least 8 weeks.

Embodiment 35. The method of embodiment 30, where the administering is following a regimen of at least 8 weeks and at least three times per week.

Embodiment 36. The method of embodiment 35, where the subject is capable of achieving a primary outcome at the end of the regimen, where the primary outcome is defined as a steroid-free clinical remission and endoscopic remission or response at the end of the treatment regimen, where the steroid-free clinical remission is defined as a total Mayo score of 2 or lower with all sub-scores of 1 or lower, where the endoscopic remission or response is defined as a reduction of at least 1 point from baseline in endoscopy score.

Embodiment 37. A method for selecting a treatment plan for treating ulcerative colitis (UC) in a subject in need thereof, the method comprising determining the level of Fusobacterium, Sutterella, or both in the subject's gut; and recommending a fecal bacteria-based therapy when the level of Fusobacterium, Sutterella, or both is below a predetermined level.

Embodiment 38. A method for selecting a treatment plan for treating ulcerative colitis (UC) in a subject in need thereof, the method comprising determining the level of one or more bacteria selected from the group consisting of Barnesiella, Parabacteroides, Clostridium IV, Ruminococcus, Blautia, Dorea, Ruminococcus2, and Clostridium XVIII in the subject's gut; and recommending a fecal bacteria-based therapy when the level of one or more bacteria selected from the group consisting of is above a predetermined level.

Embodiment 39. The method of embodiment 37 or 38, where the level of one or more bacteria is determined via analyzing said subject's feces.

Embodiment 40. A method of providing a therapeutic dosing regimen to a patient with a gastrointestinal (GI) disorder in need thereof, the method comprising administering to the patient a therapeutic composition comprising fecal microbes based upon a level of a fecal marker for intestinal inflammation.

Embodiment 41. The method of embodiment 40, wherein the GI disorder is selected from the group consisting of Antibiotic Associated Colitis, Chronic Clostridium difficile Infection (CDI), Chronic constipation, Chronic Fatigue Syndrome (CFS), Collagenous Colitis, Colonic Polyps, Constipation Predominant FBD, Crohn's Disease, Functional Bowel Disease (FBD), Gastro-oesophageal Reflux, Irritable bowel syndrome (IBS) constipation-predominant, IBS diarrhea/constipation alternating, IBS diarrhea-predominant, IBS pain-predominant, Indeterminate Colitis, Inflammatory Bowel Disease (IBD), Microscopic Colitis, Mucous Colitis, Multiple Sclerosis, Non-ulcer Dyspepsia, Norwalk Viral Gastroenteritis, Pain Predominant FBD, Primary Clostridium difficile Infection (CDI), Primary Sclerosing Cholangitis (PSC), Pseudomembranous Colitis, Small Bowel Bacterial Overgrowth, Ulcerative Colitis, and Upper Abdominal FBD.

Embodiment 42. The method of embodiment 40, wherein the gastrointestinal disorder is an inflammatory bowel disease (IBD).

Embodiment 43. The method of embodiment 40, wherein the gastrointestinal disorder is ulcerative colitis.

Embodiment 44. The method of embodiment 40, wherein the gastrointestinal disorder is Crohn's disease.

Embodiment 45. The method of embodiment 40, wherein the fecal marker for intestinal inflammation is selected from the group consisting of calprotectin, lactoferrin, M2-PK, neopterin, metalloproteinases, myeloperoxidases, and polymorphonuclear elastase.

Embodiment 46. The method of any one of embodiments 40 to 44, wherein the fecal marker for intestinal inflammation is calprotectin.

Embodiment 47. The method of embodiment 46, wherein the method comprises increasing a dosage or a dosing frequency by at least 2 times for one to ten weeks when the patient exhibits a fecal calprotectin level of above 500 μg/g.

Embodiment 48. The method of embodiment 46, wherein the method comprises
a. gradually decreasing a dosage or a dosing frequency by at least about 20% for at least 2 weeks when the patient exhibits a fecal calprotectin level of below 500 μg/g; and
b. monitoring the fecal calprotectin level in the patient.

Embodiment 49. The method of embodiment 46, wherein the method comprises decreasing a dosage or a dosing frequency by at least about 20% for at least 2 weeks when the patient's fecal calprotectin level decrease by at least 20% from a baseline level.

Embodiment 50. The method of embodiment 40, wherein the therapeutic composition comprises viable fecal microbes.

Embodiment 51. The method of embodiment 40, wherein the therapeutic composition comprises spores.

Embodiment 52. The method of embodiment 40, wherein said therapeutic composition comprises both live non-pathogenic fecal bacteria and a non-cellular fecal filtrate.

Embodiment 53. The method of embodiment 40, wherein said therapeutic composition is formulated as an enteric coated capsule or an acid-resistant capsule.

Embodiment 54. The method of embodiment 40, wherein the therapeutic dosing regimen comprises a dose from 10⁷ to 10¹⁴ cfu or total number of cells.

Embodiment 55. A method for optimizing the dosing regimen of a fecal microbe-based therapy in a patient with a gastrointestinal disorder in need thereof, the method comprising:
a. administering to the patient a therapeutic composition comprising fecal microbes at a first dosing regimen comprising a first dosage at a first dosing frequency;
b. determining the level of a fecal marker for intestinal inflammation in the patient; and
c. modifying the first dosing frequency based on the level of the fecal marker for intestinal inflammation.

Embodiment 56. The method of embodiment 55, wherein the GI disorder is selected from the group consisting of Antibiotic Associated Colitis, Chronic Clostridium difficile Infection (CDI), Chronic constipation, Chronic Fatigue Syndrome (CFS), Collagenous Colitis, Colonic Polyps, Constipation Predominant FBD, Crohn's Disease, Functional Bowel Disease (FBD), Gastro-oesophageal Reflux, Irritable bowel syndrome (IBS) constipation-predominant, IBS diarrhea/constipation alternating, IBS diarrhea-predominant, IBS pain-predominant, Indeterminate Colitis, Inflammatory Bowel Disease (IBD), Microscopic Colitis, Mucous Colitis, Multiple Sclerosis, Non-ulcer Dyspepsia, Norwalk Viral Gastroenteritis, Pain Predominant FBD, Primary Clostridium difficile Infection (CDI), Primary Sclerosing Cholangitis (PSC), Pseudomembranous Colitis, Small Bowel Bacterial Overgrowth, Ulcerative Colitis, and Upper Abdominal FBD.

Embodiment 57. The method of embodiment 55, wherein the gastrointestinal disorder is an inflammatory bowel disease (IBD).

Embodiment 58. The method of embodiment 55, wherein the gastrointestinal disorder is ulcerative colitis.

Embodiment 59. The method of embodiment 55, wherein the gastrointestinal disorder is Crohn's disease.

Embodiment 60. The method of embodiment 55, wherein the fecal marker for intestinal inflammation is selected from the group consisting of calprotectin, lactoferrin, M2-PK, neopterin, metalloproteinases, myeloperoxidases, and polymorphonuclear elastase.

Embodiment 61. The method of any one of embodiments 55 to 60, wherein the fecal marker for intestinal inflammation is calprotectin.

Embodiment 62. The method of embodiment 61, wherein the method comprising increasing the first dosage or the first dosing frequency by at least 2 times for one to ten weeks when the patient exhibits a fecal calprotectin level of above 500 μg/g.

Embodiment 63. The method of embodiment 61, wherein the method comprising
a. gradually decreasing the first dosage or first dosing frequency by at least about 20% for at least 2 weeks when the patient exhibits a fecal calprotectin level of below 500 μg/g; and
b. monitoring the fecal calprotectin level in the patient.

Embodiment 64. The method of embodiment 63, wherein the method further comprising maintaining a stable dosing regimen in the patient when the patient exhibits a fecal calprotectin level of below 50 μg/g.

Embodiment 65. The method of embodiment 63, wherein the method comprises decreasing a dosage or a dosing frequency by at least about 20% for at least 2 weeks when the patient's fecal calprotectin level decrease by at least 20% from a baseline level.

Embodiment 66. The method of embodiment 55, wherein the therapeutic composition comprises viable fecal microbes.

Embodiment 67. The method of embodiment 55, wherein the therapeutic composition comprises spores.

Embodiment 68. The method of embodiment 55, wherein said therapeutic composition comprises both live non-pathogenic fecal bacteria and a non-cellular fecal filtrate.

Embodiment 69. The method of embodiment 55, wherein said therapeutic composition is formulated as an enteric coated capsule or an acid-resistant capsule.

Embodiment 70. The method of embodiment 55, wherein the therapeutic dosing regimen comprises a dose from 10⁷ to 10¹⁴ cfu or total number of cells.

Embodiment 71. A method for selecting a fecal donor, the method comprising
a. administering to a test subject a fecal therapeutic composition derived from the fecal donor,
b. measuring a fecal marker for intestinal inflammation in the test subject,
c. selecting the fecal donor based on the level of the fecal marker for intestinal inflammation.

Embodiment 72. The method of embodiment 71, wherein the test subject is a human patient having a GI disorder or an animal model for the GI disorder.

Embodiment 73. The method of embodiment 72, wherein the GI disorder is selected from the group consisting of Antibiotic Associated Colitis, Chronic Clostridium difficile Infection (CDI), Chronic constipation, Chronic Fatigue Syndrome (CFS), Collagenous Colitis, Colonic Polyps, Constipation Predominant FBD, Crohn's Disease, Functional Bowel Disease (FBD), Gastro-oesophageal Reflux, Irritable bowel syndrome (IBS) constipation-predominant, IBS diarrhea/constipation alternating, IBS diarrhea-predominant, IBS pain-predominant, Indeterminate Colitis, Inflammatory Bowel Disease (IBD), Microscopic Colitis, Mucous Colitis, Multiple Sclerosis, Non-ulcer Dyspepsia, Norwalk Viral Gastroenteritis, Pain Predominant FBD, Primary Clostridium difficile Infection (CDI), Primary Sclerosing Cholangitis (PSC), Pseudomembranous Colitis, Small Bowel Bacterial Overgrowth, Ulcerative Colitis, and Upper Abdominal FBD.

Embodiment 74. The method of embodiment 71, wherein the fecal marker for intestinal inflammation is selected from the group consisting of calprotectin, lactoferrin, M2-PK, neopterin, metalloproteinases, myeloperoxidases, and polymorphonuclear elastase.

Embodiment 75. The method of embodiment 71, wherein the fecal marker for intestinal inflammation is calprotectin.

Embodiment 76. The method of embodiment 71, wherein the fecal donor is selected based on a response curve of the fecal marker for intestinal inflammation in said test subject.

Embodiment 77. The method of embodiment 71, wherein the fecal therapeutic composition comprises viable fecal microbes.

Embodiment 78. The method of embodiment 71, wherein the fecal therapeutic composition comprises spores.

Embodiment 79. The method of embodiment 71, wherein the fecal therapeutic composition comprises a fecal microbiota composition.

Embodiment 80. The method of embodiment 71, wherein the fecal therapeutic composition comprises a non-selected microbiota.

Embodiment 81. The method of embodiment 71, wherein the fecal therapeutic composition comprises a substantially complete microbiota.

Embodiment 82. The method of embodiment 71, wherein the fecal therapeutic composition comprises a non-cellular fecal filtrate.

Embodiment 83. The method of embodiment 71, wherein the fecal therapeutic composition comprises both live non-pathogenic fecal bacteria and a non-cellular fecal filtrate.

Embodiment 84. The method of embodiment 71, wherein said fecal therapeutic composition is formulated as an enteric coated capsule or an acid-resistant capsule.

### EXAMPLES

### Example 1. Patient Selection Criteria

Patients, including males and females aged 18 to 75 years, who have clinically and endoscopically active ulcerative colitis, with a total Mayo score of 4-10, which incorporates stool frequency, rectal bleeding, mucosal appearance at endoscopy, and physician's global assessment (PGA) are included. The endoscopy score has to be ≥1 and PGA score ≤2. Furthermore, such ulcerative colitis has to be present for more than three months in duration. Ulcerative colitis of any extent is treated except for isolated proctitis that are <5 cm. See Table 4 for all inclusion criteria.

TABLE 4
Study Participant Inclusion Criteria
Patients must meet all the following INCLUSION CRITERIA at enrollment to be eligible to participate

1. Males and females aged 18 to 75 years, inclusive
2. Ulcerative colitis >3 months duration
3. Ulcerative Colitis of any extent except isolated proctitis <5 cm
4. Currently active mild-moderate ulcerative colitis, as measured by a Mayo score of 4-10, which
incorporates stool frequency, rectal bleeding, mucosal appearance, and physician's assessment as a four
point category score. Endoscopy score must be ≥1 and physician global assessment score ≤2
5. Provide written informed consent to participate as shown by a signature on the consent form

Patients receiving treatment with oral 5-aminosalicylates, thiopurines and methotrexate has to be at stable doses. Oral prednisolone is allowed in patients, provided the dose is stable proceeding enrollment. Patients having undergone a mandatory oral prednisolone may taper of up to 2.5 mg per week, and need to be steroid-free by week 8.

Patients receiving rectal therapies in the past 2 weeks, receiving antibiotics or probiotics in the past 4 weeks, and receiving biologic therapy in the past 12 weeks are to be excluded. Patients exhibiting evidence or history of toxic megacolon, as well as any other significant gastrointestinal conditions, including but not limited to irritable bowel syndrome, diverticulitis, and neoplasm, are also excluded. Patients being diagnosed of Crohn disease or indeterminate colitis are excluded. Patients with perianal disease such as fistulae and pre-existing fissures are excluded. Patients with severe anaemia, leucopaenia, or granulocytopenia are excluded. Patients who had appendectomy less than 3 months prior to treatment are also excluded. Patients with significant food hypersensitivity are excluded. See Table 5 for all exclusion criteria.

TABLE 5
Study Participant Exclusion Criteria
Patients must not meet any of the following EXCLUSION CRITERIA at enrolment to be eligible to participate

```
1. Consent not obtained or unable to give informed consent
2. Unable to communicate with the investigators and comply with the study requirements
3. Females who are pregnant or actively trying to fall pregnant
4. Patients unwilling to practice an effective method of contraception throughout the study period
5. Patients defined as in remission by the investigator
6. Patients with mild ulcerative colitis (Mayo score < 4)
7. Patients with severe ulcerative colitis (Mayo score > 10)
8. Evidence or history of toxic megacolon
9. Isolated proctitis <5 cm
10. A diagnosis of Crohn's Disease or indeterminate colitis
11. Patients with perianal disease (e.g. fistulae, pre-existing fissures)
12. Severe anaemia, leucopaenia or granulocytopenia
13. Detection of a gastrointestinal pathogen on stool analysis
    Need to exclude /treat active GI infection before inclusion into study (e.g. giardia, C. diff, CMV etc.)
    Prior GI infection is not an exclusion to enrolment as long as successful treatment and eradication is documented
14. Constipation-predominant Ulcerative Colitis with <3 bowel motions/day
15. Any other significant GI condition e.g. Irritable bowel syndrome, diverticulitis, neoplasm etc.
16. Significant gastrointestinal surgery e.g. colon resection, colectomy
    Minor gastrointestinal surgery will be reviewed on a case by case basis by the investigator
    Regarding appendicectomy, only exclude patients who had appendicectomy <3 months ago
17. Patients taking antimicrobials (antibiotics, antifungals, antivirals) for any reason, including antibiotics
for ulcerative colitis, in the preceding four weeks
18. Patients who are steroid dependent and requiring >20 mg prednisone or >9 mg budesonide daily at the
time of enrolment
19. Patients who have recently taken or are actively taking or expected to require prohibited medication/s
during the study period including follow-up. These include
    Treatment with anti-tumour necrosis factor agents e.g. infliximab, adalimumab, within the last 12 weeks
    Treatment with other major immunosuppressant agents including calcineurin inhibitors,
    mammalian target of rapamycin (mTOR) inhibitors, chemotherapeutic anti-neoplastic agents,
    lymphocyte depleting biological agents within the last 12 weeks
    Probiotic therapy in the last 4 weeks
    Experimental / trial drug protocol involvement in last 12 weeks
    Anti-mycobacterial (TB or MAC) therapy in last 4 weeks
20. Clinical evidence of any major, co-morbid chronic disease that may interfere with the patient's ability to
enter the trial. Patients with a concomitant illness sufficiently severe as to jeopardize participation in the
study or interpretation of results will be excluded from the study
    In particular, severe immunodeficiency including but not limited to decompensated liver
    cirrhosis, advanced HIV/AIDS and recent bone marrow transplant will be an absolute
    contraindication to FMT
21. Patients with food hypersensitivity deemed by the investigator to be significant during the trial e.g., nut allergy
22. Patients who have travelled overseas to an infectious diarrhea endemic area within the last month or
have overseas travel planned during the study period (not feasible to be compliant with enema therapy)
```

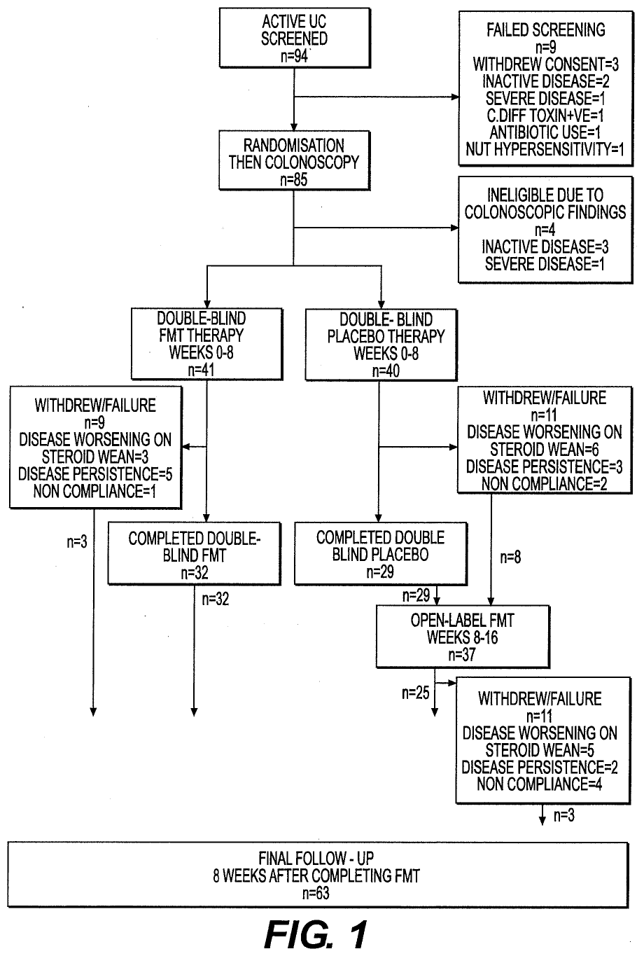

Based on the above criteria, eighty-five patients are recruited in this study. The selected patients are randomized with 81 commencing treatment (FIG. 1). The two groups are well matched except for disease severity; significantly more patients with the mildest (Mayo 1) endoscopic disease are randomized to placebo. Table 6 summarizes the baseline characteristics of the recruited patients.

TABLE 6
Baseline Patient Characteristics.

| | FJIT (n = 41) | Placebo (n = −40) | P Value |
|---|---|---|---|
| Age, y | 35.5 (27.8-48.9) | 35.4 (27.7-45.6) | 0.97 |
| Male Sex, n (%) | 22 (54%) | 25 (63%) | 0.42 |
| Caucasian race, n (%) | 27 (66%) | 27 (68%) | 0.88 |
| Non Smoker, n (%) | 23 (56%) | 21 (53%) | 0.75 |
| UC <1 year, n (%) | 2 (5%) | 2 (5%) | 0.98 |
| Disease Duration, y | 5.8 (3.4-9.0) | 5.8 (2.7-9.4) | 0.55 |
| Disease Extent, n (%) | | | |
| Proctitis | 4 (10%) | 8 (20%) | 0.19 |
| Left Sided Colitis | 28 (68%) | 20 (50%) | 0.09 |
| Pancolitis | 9 (22%) | 12 (30%) | 0.41 |
| Concomitant Medications, | | | |
| n (%) | | | |
| Nil | 9 (22%) | 6 (15%) | 0.42 |
| Oral 5-ASA | 26 (63%) | 28 (70%) | 0.53 |
| Oral Immunomodulator | 20 (49%) | 15 (38%) | 0.31 |
| Oral Steroids | 9 (22%) | 11 (28%) | 0.56 |
| Prior Anti-TNIF Therapy, | 9 (22%) | 6 (15%) | 0.42 |
| n (1%) | | | |
| Prior Other Biologic | 2 (5%) | 0 (0%) | 0.16 |
| Therapy, n (%) | | | |
| Mayo Score | 8 (6-9) | 8 (6-9) | 0.43 |
| Mayo Endoscopic Subscore, | | | |
| n (%) | | | |
| Mayo 1 | 1 (2%) | 7 (18%) | 0.02* |
| Mayo 2 | 27 (65%) | 15 (38%) | 0.01* |
| Mayo 3 | 13 (32%) | 18 (45%) | 0.22 |
| UCEIS Score | 4 (3.5-5.5) | 4 (3-5) | 0.76 |
| IBDQ Score | 123 (99-157) | 119 (109-149) | 0.78 |
| Faecal Calprotectin, ug/g | 705 (226-1220) | 505 (193-1475) | 0.41 |
| Erythrocyte Sedimentation | 14 (5.5-29.5) | 10 (5-20) | 0.20 |
| Rate, mm/hr | | | |
| C-Reactive Protein, mg/L | 2.6 (1.0-7.1) | 2.9 (0.8-5.8) | 0.93 |
| White Cell Count. ×10⁹/L | 7.8 (6.2-9.7) | 8.0 (6.3-9.9) | 0.71 |
| Neutrophil Count, ×10⁹/L | 4.8 (3.5-6.9) | 5.7 (3.7-6.7) | 0.60 |
| Haemoglobin, g/L | 134 (129-143) | 136 (127-148) | 0.56 |
| Platelet Count, ×10⁹/L | 299 (248-352) | 306 (251-362) | 0.48 |
| Albumin, g/L | 46 (43-48) | 45 (43-48) | 0.50 |

### Example 2. Stool Donors Selection

Stool donors, including males and females aged 18 to 65 years, who have no history or current symptoms of gastrointestinal disease including but not limited to inflammatory bowel disease and irritable bowel syndrome are included. Donors should not have any major active medical co-mobidities. Donors should have minimal regular medications with no medications that may interfere with stool viability, including no antimicrobials, probiotics and proton pump inhibitors in the preceding three months prior to donation. See Table 7 for all donor inclusion criteria.

TABLE 7
Healthy Fecal Donor Inclusion Criteria
Donors must meet all the following INCLUSION CRITERIA at enrolment

```
1. Males and females aged 18 to 65 years, inclusive
2. No history or current symptoms of gastrointestinal disease including but not limited to inflammatory
bowel disease (IBD) and irritable bowel syndrome (IBS)
3. No other major active medical co-morbidities
4. Minimal regular medications with no medications that may interfere with stool viability
    including no antimicrobials (antibiotics, antivirals, antifungals), probiotics and proton pump
    inhibitors (PPIs) in the preceding 3 months
5. Provide written informed consent to participate as shown by a signature on the consent form
```

To exclude unhealthy donors, a potential donor's stool is evaluated using one or more of the following tests: Clostridium difficile toxin PCR, fecal microscopy/culture/sensitivity with routine bacterial culture for enteric pathogens, fecal Giardia antigen, fecal Cyrptosporidium antigen, fecal ova/cysts/parasites (including Blastocystis hominis and Dientamoeba fragilis), and Norovirus EIA. A potential donor's blood is also tested for one or more of the following: complete blood count (CBC); electrolytes, urea and creatinine (EUC); liver function tests (LFT); erythrocyte sedimentation rate (ESR); C-Reactive protein (CRP); human immunodeficiency virus (HIV) type 1 and 2; Hepatitis A virus IgM; Hepatitis B virus surface antigen, Hepatitis B virus core antibody (IgM+IgG), Hepatitis B virus surface antibody; Hepatitis C virus antibody; Rapid plasma regain and/or fluorescent treponemal antibody-absorbed; and human T-cell lymphotropic virus (HTLV) 1 and 2.

TABLE 8
Healthy Fecal Donor Exclusion Criteria
Donors must not meet any of the following EXCLUSION CRITERIA at enrolment

```
1. Risk of infectious agent
    Known HIV, hepatitis B or hepatitis C infection
    Known exposure to HIV or viral hepatitis within the previous 12 months
    High risk sexual behavior (e.g. sexual contact with anyone with HIV/AIDS or viral hepatitis, men
    who have sex with men, sex for drugs or money)
    Use of illicit drugs
    Tattoo or body piercing within the preceding 6 months
    Incarceration or history of incarceration
    Known current communicable disease (e.g. upper respiratory tract infection)
    Risk factors for variant Creutzfeldt-Jakob disease
    Travel within last 2 weeks to areas of the world where diarrheal illnesses are endemic or risk of
    traveler's diarrhea is high
2. Gastrointestinal co-morbidities
    History of or current inflammatory bowel disease (IBD)
    History of or current irritable bowel syndrome (IBS), chronic constipation, chronic diarrhea or other
    intrinsic gastrointestinal illness / condition
    History of or current gastrointestinal malignancy or known polyposis or strong family history of
    colorectal cancer
    History of major gastrointestinal surgery (e.g. gastric bypass, partial colectomy)
3. Factors that can affect the composition of the intestinal microbiota
    Antimicrobials (antibiotics, antivirals, antifungals), probiotics or proton pump inhibitors (PPIs)
    within the preceding 3 months
    Major immunosuppressive medications (e.g. calcineurin inhibitors, biological agents, exogenous
    glucocorticoids)
    Systemic anti-neoplastic agents
    Household members with active GI infection
4. Other conditions
    Systemic autoimmunity (e.g. multiple sclerosis, connective tissue disease)
    Atopic disease (e.g. moderate - severe asthma, eosinophilic disorders of the gastrointestinal tract)
    Metabolic syndrome, obesity (BMI >30) or moderate to severe under-nutrition / malnutrition
    Chronic pain syndromes (e.g. chronic fatigue syndrome, fibromyalgia) or neurologic /
    neurodevelopmental disorders
    History of malignant illness or ongoing oncologic therapy
```

Based on the above criteria and tests, 14 donors are selected.

### Example 3. FMT and Placebo Preparation and Storage

FMT infusions are constituted from the blended stool of 3 to 7 donors, to increase microbial heterogeneity. Each patient receives all their FMT infusions from the same donor batch to ensure consistency and reproducibility of the infused fecal microbiota.

Placebo infusions comprise isotonic saline. Odorant, brown food colour disodium 4,4′-2,4-dihydroxy-5-hydroxymethyl-1,3-phenylene-bisazodi-1-napthalene sulfonate (to replicate fecal odour and colour respectively), and glycerol cryoprotectant (concentration 10%) are added to the 150 ml placebo and FMT infusions, which are then stored at −80° C.

Three to seven of the selected donors contributes to each of the 21 FMT batches in the study. The use of multiple donors for all infusions is one of the features of this study.

### Example 4. Study Design

At three clinical centers patients are randomized 1:1 double-blind to FMT:placebo using permutated blocks of 4, stratified for study site and concomitant corticosteroid use.

After full bowel preparation, colonoscopy is performed to the terminal ileum and the initial infusion administered. Patients then self-administer enemas 5 times per week for 8 weeks. After 8 weeks mucosal inflammation is assessed with sigmoidoscopy.

After the initial 8 week study period placebo-treated patients are offered 8 weeks of open-label FMT enemas 5 times per week, without initial colonoscopic infusion. Sigmoidoscopy is repeated after open-label FMT.

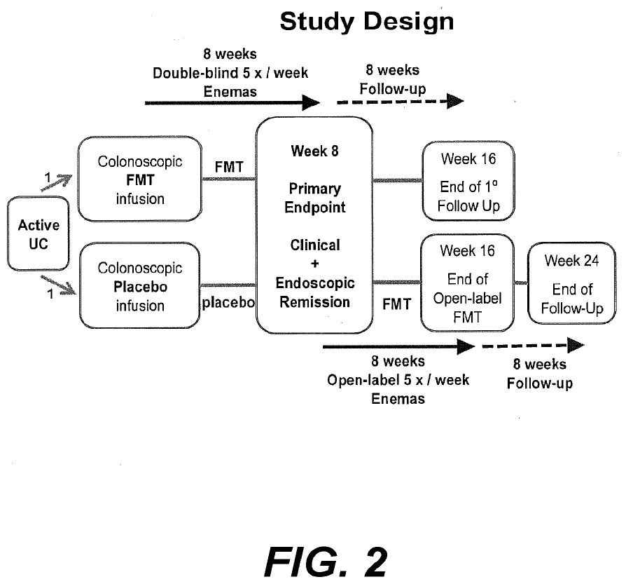

FIG. 2 shows a graphical representation of this study design.

### Example 5. Study Assessments and Endpoints

Patients are reviewed fortnightly during blinded and open label study periods with a final review 8 weeks post FMT. Blood and stool investigations are performed every 4 weeks during study therapy. Blood tests include CBC, EUC, LFT, ESR, and CRP. Stool tests include fecal calprotectin.

Evaluation of the site of worst inflammation at each endoscopy, using the Mayo endoscopy sub-score and UCEIS score, is undertaken with blinded review and central consensus scoring of all endoscopic photo images by 5 IBD-expert gastroenterologists.

The primary composite outcome is steroid-free clinical remission together with endoscopic remission or response at week 8, defined as a total Mayo score of ≤2 with all sub-scores ≤1 and ≥1 point reduction from baseline in endoscopy score.

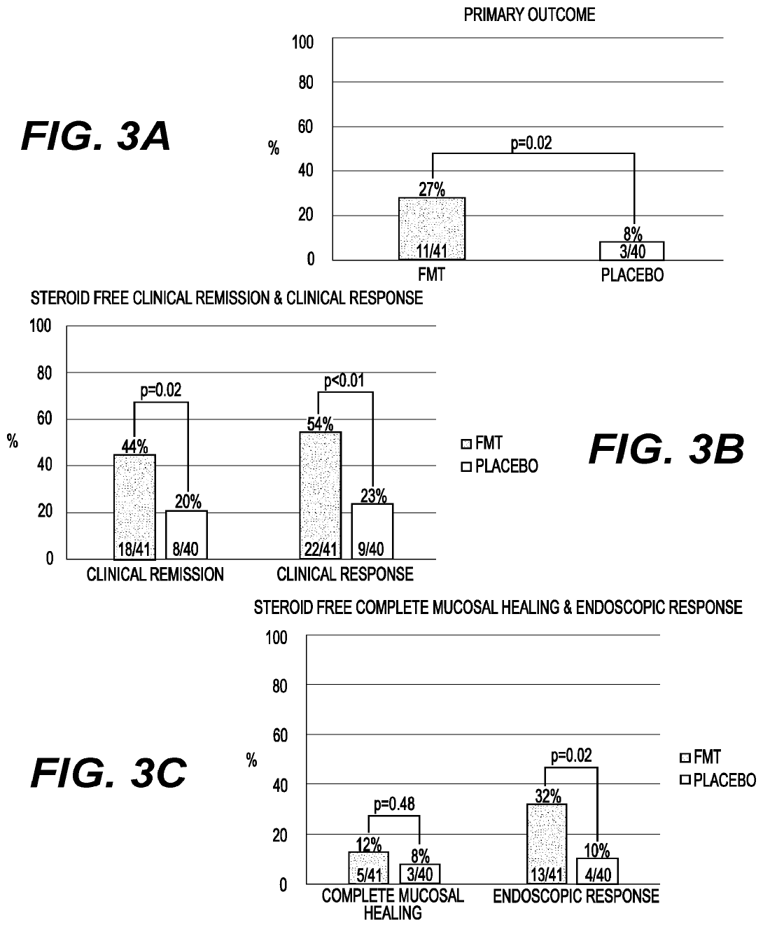

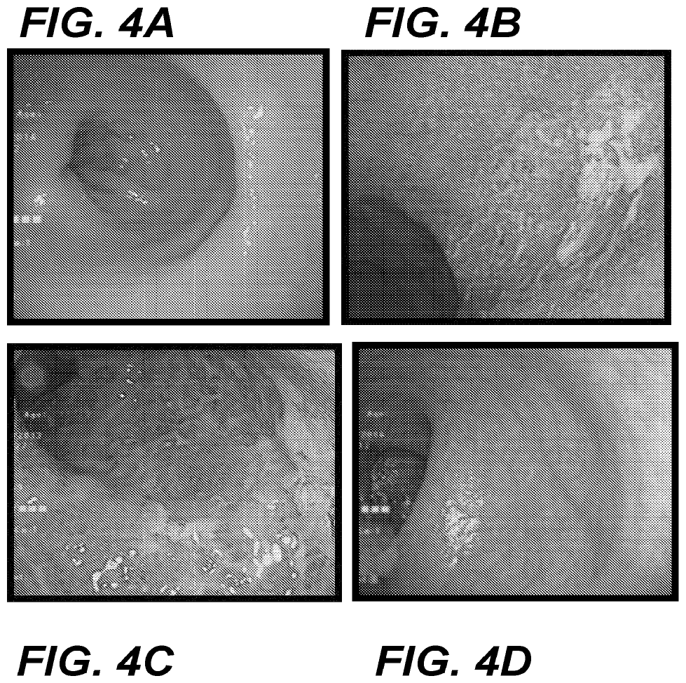

Eleven of 41 (27%) FMT-treated patients and 3 of 40 (8%) placebo-treated patients achieve the primary outcome (P=0.02, OR 4.5 (95% CI 1.2-17.7)) (FIG. 3A, FIG. 4A-D).

FIG. 3A shows the number of patients in the FMT and placebo-treated groups who achieved the primary outcome of steroid-free clinical remission and endoscopic remission or response (total Mayo score ≤2 with all sub-scores ≤1 and ≥1 point reduction from baseline in endoscopy sub-score) at week 8. The total Mayo score can range from 0 to 12, and sub-scores range from 0 to 3, with higher scores indicating more severe disease.

FIG. 4A and FIG. 4B show the effect of a FMT therapy in a 37 year old female patient with a 4 year history of left sided ulcerative colitis and acute colitis (diarrhea 6 times per day with bleeding) despite maximal oral and topical 5-ASA therapy. FIG. 4A shows an exemplary baseline endoscopic appearance of 25 cm recto-sigmoid active colitis with endoscopic Mayo sub-score 2, and total Mayo score 8. FIG. 4B shows an exemplary endoscopic appearance in the same patient at the end of week 8 blinded FMT therapy with endoscopic Mayo sub-score 0, and total Mayo score 0. This patient remains in clinical remission at final study follow up 8 weeks after completing blinded FMT therapy.

FIG. 4C and FIG. 4D show the effect of a FMT therapy in a 28 year-old female patient with a 7 year history of extensive ulcerative colitis. This patient experiences failed therapy with mesalamine, probiotic and adalimumab. Accordingly, the patient is maintained on azathioprine and allopurinol, and was steroid-dependent on oral budesonide 9 mg/day. At study entry this patient has diarrhea 8 times per day with bleeding and abdominal pain. FIG. 4C shows an exemplary baseline endoscopic appearance of extensive colitis to the hepatic flexure with endoscopic Mayo sub-score 3, and total Mayo score 10. This patient receives placebo treatment during the primary study, but was unable to taper corticosteroids, and was therefore a treatment failure in the primary outcome. FIG. 4D shows Endoscopic appearance in the same patient at the completion of 8 weeks open-label FMT, with endoscopic Mayo sub-score 0 and Total Mayo Score 0. After 8 weeks of open-label FMT this patient has weaned corticosteroids completely and is in clinical and endoscopic remission.

Secondary outcomes include steroid-free clinical remission (combined score ≤1 for rectal bleeding plus stool frequency Mayo sub-scores), clinical response (decrease ≥3 and/or ≥50% reduction from baseline in combined rectal bleeding plus stool frequency Mayo sub-scores), endoscopic response (Mayo endoscopy sub-score ≤1 with a reduction ≥1 from baseline), complete mucosal healing (Mayo endoscopy sub-score 0), quality of life using IBDQ¹⁰ and safety.

Blinded central reading of all endoscopic images is performed using both Mayo and UCEIS scoring. Assessing steroid free endoscopic outcomes using a decrease ≥3 points and/or ≥50% reduction from baseline in UCEIS score, the difference between the FMT and placebo arms at week 8 was 37% vs. 10%, p<0.01, OR 5.2, 95% CI 1.5-17.5. When the criteria of UCEIS≤1 is used, the difference between FMT and placebo treated patients was 17% vs. 8%, p=0.19, OR 2.5, 95% CI 0.6-10.6) at week 8.

Significant differences are observed in the total Mayo score, and in the decrease in total Mayo score, at week 8, between the FMT and placebo treated groups (Table 9). IBDQ FMT data is available only from 31 patients. All missing data is assigned worst value in the entire cohort. All continuous variables are reported as median and interquartile range.

TABLE 9
Week 8 Efficacy Outcomes.

| | FMT | Placebo | |
|---|---|---|---|
| | (n = 41, | (n = 40, | |
| Adjusted Outcome Measure | data on 32) | data on 29) | P Value |
| Total Mayo Score at 8 weeks | 4 (2-6) | 7 (4-9) | 0.01 |
| Total Mayo Score decrease | 4 (2.3-6.0) | 1 (−0.5-2) | <0.01 |
| at 8 weeks | | | |
| IBDO at 8 weeks | 182 (135-206) | 151 (130-195) | 0.21 (NS) |
| IBDCI Improvement | 40 (15-70) | 23 (10-33) | 0.13 (NS) |
| at 8 weeks | | | |
| IBDC/ increase of 32 | 18 (44%) | 9 (23%) | 0.04 |
| points at 8 weeks | | | |
| CRP | 2.8 (1.0-1.3) | 3.0 (1.7-4.9) | 0.65 (NS) |
| ESR | 17 (6.5-21) | 11 (6-20) | 0.85 (NS) |
| Calprotectin | 335 (91-1150) | 410 (157-1345) | 0.55 (NS) |

FIG. 3B shows the number of patients in steroid-free clinical remission (combined score of ≤1 for rectal bleeding plus stool frequency Mayo subscores) and clinical response (decrease ≥3 points and/or ≥50% reduction from baseline in the combined score for rectal bleeding plus stool frequency Mayo subscores) at week 8. Steroid-free clinical remission (44% vs. 20%, P=0.02, OR 3.1, 95% CI 1.2-8.4) and steroid-free clinical response (54% vs. 23%, P<0.01, OR 4.0, 95% CI 1.5-10.4) at week 8 is significantly greater in FMT than placebo-treated patients.

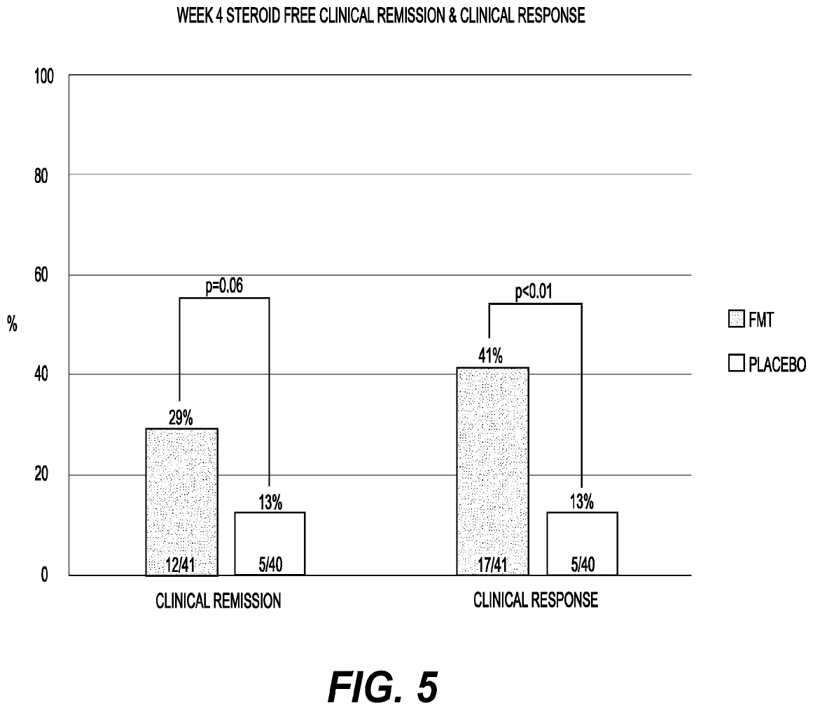

FIG. 3C shows the number of patients with steroid-free endoscopic response (Mayo endoscopy sub-score ≤1 with a reduction ≥1 from baseline) and complete mucosal healing (Mayo endoscopy sub-score 0). At week 8 steroid-free endoscopic response (32% vs 10%, P=0.02, OR 4.2, 95% CI 1.2-14.2) is significantly greater in the FMT-treated patients. Complete mucosal healing (Mayo 0: 12% vs. 8%, P=0.48, OR 1.7 95% CI 0.4-7.7) is greater in the FMT than placebo arms but this difference was not significant. Outcomes are similar with UCEIS scoring (FIG. 5). FIG. 5 shows the speed of onset of therapy. At the end of week 4 clinical response is significantly greater in FMT [17 of 41 (41%)] than placebo [5 of 40 (13%)] treated patients (p<0.01, OR 5.0, 95% CI 1.6-15.3]. Clinical remission does not differ significantly between treatment arms [12 of 41 (29%) vs 5 of 40 (13%) respectively (P=0.06, OR 2.9, 95% CI 0.91-9.2)].

IBDQ score and inflammatory markers do not differ significantly between groups (Table 9).

Thirty seven initially placebo-treated patients proceed to open-label FMT. After open-label FMT, 10 (27%) meet the primary endpoint, 17 (46%) are in clinical remission and 8 (22%) have complete mucosal healing.

No relationship between outcome and anatomical disease extent is observed (P=0.23). The severity of endoscopic inflammation is associated with therapeutic outcomes (p=0.01) with no patient with Mayo endoscopy score 3 at study entry achieving the primary outcome. Corticosteroid use is also associated with therapeutic outcome (p=0.02) with no patient entering the study on corticosteroids achieving the primary endpoint at the end of blinded therapy; one patient on corticosteroids at open-label FMT entry meets the primary endpoint at completion.

Sixty-three patients attend a final study follow-up 8 weeks after completing double-blind or open-label FMT, of whom 28 are in clinical remission and 20 require UC therapy escalation.

Nine patients on blinded FMT and 11 on placebo (P=0.56) withdraw or have protocol failure prior to week 8. The reasons for withdrawal or protocol failure on blinded therapy include disease worsening on steroid wean (3 FMT, 6 placebo), disease persistence or worsening in the absence of steroid wean (5 FMT, 3 placebo) and non-compliance (1 FMT, 2 placebo).

Eleven patients who commence open label FMT either withdraw or have a protocol failure: 5 have disease worsening on steroid wean, 2 have disease persistence or worsening in the absence of steroid wean, 4 are non-compliant.

Thirty two (78%) FMT and 33 (83%) placebo-treated patients experience at least one adverse event during blinded therapy, with no significant difference in number or type of adverse events (Table 10). The most common adverse events are self-limiting gastrointestinal complaints (abdominal pain, bloating, and flatulence). Six serious adverse events (SAEs) occur during study therapy: 2 blinded FMT, 1 placebo, 3 open label FMT. One patient with refractory colitis on blinded FMT withdraw at week 2 due to clinical and endoscopic (Mayo 2 to 3, UCEIS 5 to 7) deterioration, and underwent colectomy. One patient with moderate to severe colitis remain unwell at week 3 of blinded active therapy, withdraw and is hospitalized for intravenous corticosteroid therapy. One patient with moderate to severe colitis is withdrawn at week 3 of placebo therapy and require hospitalization. Three initial placebo-treated patients fail to improve with open-label FMT and were hospitalized for escalation of therapy.

TABLE 10
Adverse Events.

| | FMT | Placebo | | Open Label | Follow Up |
|---|---|---|---|---|---|
| Adverse Event | (n = 41) | (n = 40) | P Value | (n = 37) | (n = 63) |
| Total AE | 78 | 80 | 0.78 (NS) | 35 | 14 |
| Total Patients with AE | 32 (78%) | 33 (83%) | 0.62 (NS) | 18 (49%) | 9 (14%) |
| Total infection Related AE | 11 | 17 | 0.22 (NS) | 9 | 3 |
| Total Patients with Infection | 10 (24%) | 14 (35%) | 0.30 (NS) | 8 (22%) | 3 (5%) |
| Related AE | | | | | |
| Abdominal pain | 12 (29%) | 11 (28%) | 0.86 (NS) | 5 (14%) | 1(2%) |
| Colitis | 10 (24%) | 9 (23%) | 0.84 (NS) | 3 (8%) | 4 (6%) |
| Flatulence | 10 (24%) | 8 (20%) | 0.64 (NS) | 2 (5%) | |
| Bloating | 8 (20%) | 11 (28%) | 0.40 (NS) | 3 (8%) | |
| Upper Respiratory Tract | 7 (17%) | 6 (15%) | 0.80 (NS) | 4 (11%) | 2(3%) |
| Infection | | | | | |
| Headache | 4 (10%) | 2 (5%) | 0.41 (NS) | 2 (5%) | |
| Dizziness | 3 (7%) | 3 (8%) | 0.97 (NS) | — | |
| Fever | 3 (7%) | 2 (5%) | 0.67 (NS) | — | |
| Rash | 3 (7%) | — | | — | |
| Nausea | 2 (5%) | 5 (13%) | 0.22 (NS) | 1 (3%) | |
| ALT elevated | 2 (5%) | 2 (5%) | 0.98 (NS) | — | 1 (2%) |
| Chills | 2 (5%) | 2 (5%) | 0.98 (NS) | — | 1 (2%) |
| Vomiting | 2 (5%) | 1 (3%) | 0.57 (NS) | — | |
| Back pain | 2 (5%) | — | — | | |
| Flu like symptoms | 1 (2%) | 4 (10%) | 0.16 (NS) | 3 (8%) | |
| Enterocoritis | 1 (2%) | 3 (8%) | 0.29 (NS) | — | |
| Diarrhoea | 1 (2%) | — | | 1 (3%) | |
| Fracture (foot) | 1 (2%) | — | | — | |
| Reflux symptoms | 1 (2%) | — | | — | |
| Sinusitis | 1 (2%) | — | | — | |
| Haemorrhoids | 1 (2%) | — | | — | |
| Elective Surgical procedure | 1 (2%) | — | | — | |
| Anxiety | — | 1 (3%) | | 1 (3%) | 1 (2%) |
| Lung infection | — | 1 (3%) | | 1 (3%) | |
| Anal Fissure | — | 1 (3%) | | — | |
| Faecal Incontinence | — | 1 (3%) | | — | |
| Fatigue | — | 1 (3%) | | — | |
| Genital herpes | — | 1 (3%) | | — | |
| Irritability | — | 1 (3%) | | — | |
| Lip Infection | — | 1 (3%) | | — | |
| Otitis media | — | 1 (3%) | | — | |
| Sore throat | — | 1 (3%) | | — | |
| Urticarial | — | 1 (3%) | | — | |
| Arthralgia | — | — | | 1 (3%) | |
| AST elevated | — | — | | 1 (3%) | |
| Blurred vision | — | — | | 1 (3%) | |
| Depression | — | — | | 1 (3%) | |
| Dry skin | — | — | | 1 (3%) | |
| Insomnia | — | — | | 1 (3%) | |
| Myalgia | — | — | | 1 (3%) | |
| Palpitations | — | — | | 1 (3%) | |
| Productive cough | — | — | | 1 (3%) | |
| Anaemia | — | — | | — | 1 (2%) |
| Non Elective Surgical | — | — | | — | 1 (2%) |
| Procedure (intraoperative | | | | | |
| Soft tissue infection (Axillary | — | — | | — | 1 (2%) |
| abscess) | | | | | |
| Tremor | — | — | | — | 1 (2%) |
| Total SAE | 2 (5%) | 1 (3%) | 0.57 (NS) | 3 (8%) | 1 (2%) |

Six serious adverse events are observed during study therapy: 2 blinded FMT, 1 placebo, 3 open-label FMT. One patient with refractory UC on blinded FMT is withdrawn due to clinical and endoscopic deterioration, and underwent colectomy. Three initial placebo-treated patients fail to improve with open-label FMT and require hospitalization for intravenous corticosteroids or anti-TNF therapy.

Multi-donor, intensive-dosing FMT in UC appear to be safe in the short term. Most serious adverse events relate to either corticosteroid-dependent or refractory patients unable to tolerate steroid wean, or patients with moderate to severe colitis. The patient who had undergone a colectomy while on FMT demonstrates that a small subset of UC patients may be susceptible to disease worsening with this therapy.

No individual donor or donor batch is significantly associated with the primary outcome, although this particular study is not powered to evaluate this. One of the donor tends to be associated with benefit, 37% of patients with and 18% without this donor achieving the primary outcome (P=0.054). Donor batch does not correlate with the primary endpoint or serious adverse events.

Based on available data and anecdotal experience, the predicted FMT remission rate is 60%, placebo rate is 15%, and dropout rate is 30%. Forty patients per group are required for an 80% probability of demonstrating a difference with a two-sided alpha of 0.05 on intention-to-treat analysis.

All analyses are intention to treat (ITT), including all patients who received at least one study dose. Patients who require increased therapy, breach study protocol, fail to cease corticosteroids by week 8, or terminate the study for any reason are deemed treatment failures. Missing and incomplete data are assigned the worst value in the cohort for statistical analyses.

Descriptive statistics are computed for all variables. Normally distributed continuous data are expressed as mean and standard deviation, and are analyzed using unpaired t-test. Data not normally distributed are expressed as median and interquartile range and are analyzed using Wilcoxon rank sum test. Categorical data are assessed by Chi-square and Fisher's exact tests. Results are expressed as odds ratios with 95% confidence intervals. A p-value of <0.05 is considered significant.

Statistical analyses are performed using SPSS version 23.0 software (Chicago, Ill.).

### Example 6. Gastrointestinal Microbiota Analyses

Microbiological analyses are performed on patient, individual donor and FMT batch fecal samples. Samples are stored at −80° C. Fecal bacterial DNA is extracted. The 16S rRNA gene fragment is amplified using the F27 and 519R primers, then is subjected to high throughput sequencing on an Illumina MiSeq platform (2×300 bp chemistry) to determine microbiota diversity and abundance. Raw sequences are analyzed using MOTHUR (Schloss et al. Appl. Environ. Microbiol. 2009; 75:7537-41). Statistical tests are performed on counts and relative abundances.

Diversity (α- and phylogenetic) analyses and statistical analyses including principal component analysis (PCA), CLUSTER with SIMPROF testing, permutational MANOVA (PERMANOVA), and PERMDISP are performed on the reads using MOTHUR and Primer-E (Clarke. J. of Ecology 1993; 18(1):117-43). Linear Discriminant Analysis Effect Size (LEfSe) analysis (Segata et al. Genome Biol. 2011; 12:R60) is performed using the Galaxy web application (Goecks et al. Genome Biol. 2010; 11:R86).

Fecal samples are collected from 70 patients. Three hundred and fourteen patient and 113 donor fecal samples (55 individual donor and 58 batch samples) are analyzed. The number of clean sequences obtained per sample is 26976±540 Rarefaction curves suggest that sampling has reached saturation.

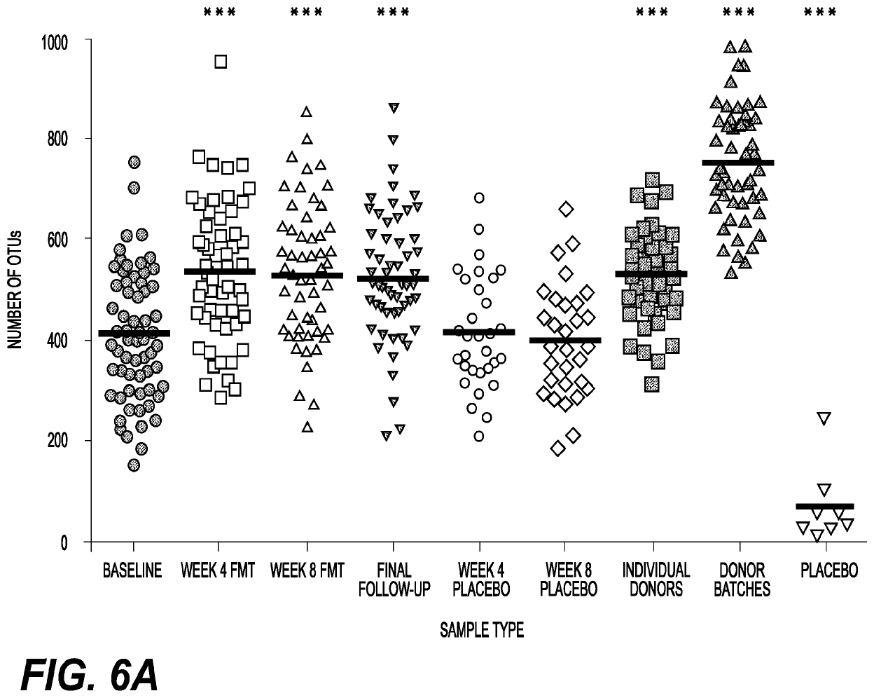

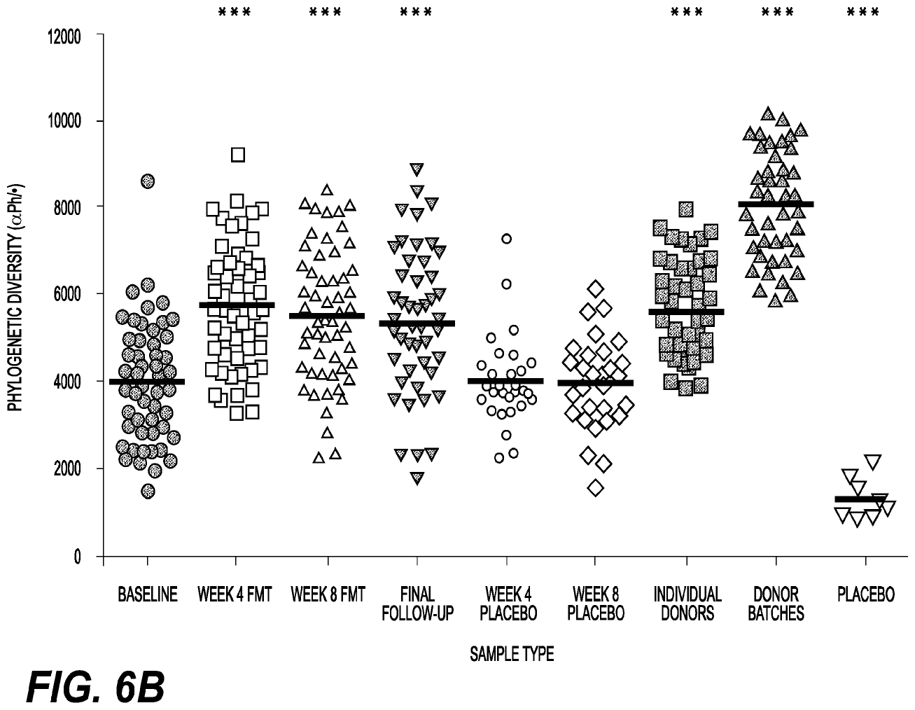

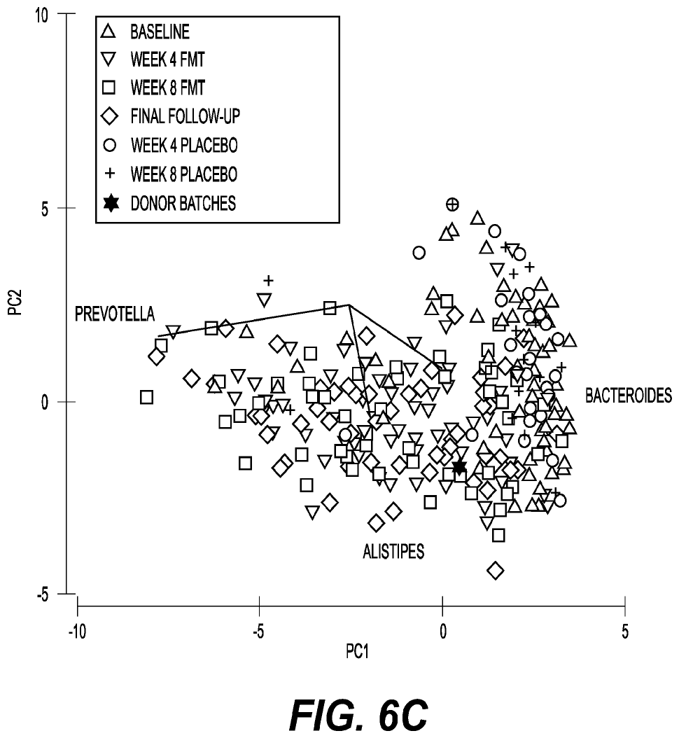

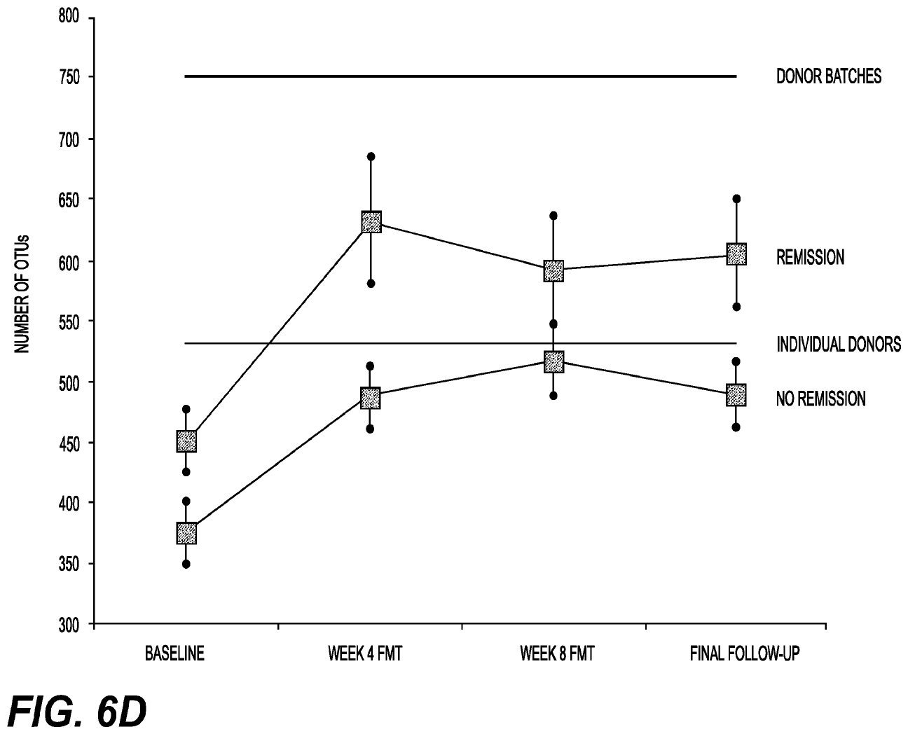

The number of operational taxonomic units (OTUs) and phylogenetic diversity are significantly higher in donor batches than individual donors (FIG. 6A and FIG. 6B). The number of OTUs and phylogenetic diversity of donor samples (batch and individual) are significantly higher than baseline patient samples (FIG. 6A and FIG. 6B). ***in FIG. 6A and FIG. 6B denotes P<0.0001.

OTU number and phylogenetic diversity increase significantly relative to baseline in all FMT-treated patients at 4 and 8 weeks (p<0.0001) and persist 8 weeks post-FMT (p<0.0001) (FIG. 6A and FIG. 6B). Similar patterns are observed for species richness and Shannon's diversity.

Significant differences in microbial profiles and reduced dispersion levels are observed from OTU to Class taxonomic levels following FMT. PCA confirms the changes in microbial profiles of patients undergoing FMT (FIG. 6C). Patient profiles shift from a dominance of Bacteroides to Prevotella (FIG. 6C). The shift in microbial profiles of patients undergoing FMT towards the donor is most notable at the OTU level.

Patient baseline samples are compared with week 4, week 8, and 8 weeks post-FMT to identify taxa altered by FMT, and with donor samples to identify OTUs associated with donor batches and those associated with the patient. Two hundred and ninety-five microbial taxa across all taxonomic levels are transplanted with FMT, of which 78 show strong associations (LDA score >3). There is a decrease in patient Bacteroides (e.g. OTU 8, 15, 69) and a marked increase in donor Prevotella (e.g. OTU 2) and donor Bacteroides (e.g. OTU 12, 26, 56) with FMT, independent of clinical outcome. This pattern is more apparent when OTUs are picked at higher resolution.

Blinded FMT-treated patients who achieve the primary outcome tend to have higher baseline alpha-diversity than those who do not (P=0.1, FIG. 6D). Blinded FMT-treatment is associated with significantly increased diversity in all patients; however patients who achieve the primary outcome have greater diversity during FMT and 8 weeks post-FMT, achieving levels higher than individual donors though lower than the donor batches (FIG. 6D). Increased α-diversity is specific to FMT; three patients who meet the primary outcome on placebo show no change in diversity.

To identify microbial taxa associated with primary outcome on FMT, LEfSe analyses are performed with blinded FMT and open-label FMT patients are stratified. 87 taxa are significantly associated with primary outcome in blinded patients and 46 taxa in open label FMT patients. A range of microbial taxa are associated with remission in the blinded FMT (e.g. Barnesiella, Parabacteroides, Clostridium IV and Ruminococcus) and open label FMT patients (e.g. Blautia, Dorea, Ruminococcus2, and Clostridium XVIII). Both Fusobacterium and Sutterella are consistently associated with lack of remission in both blinded and open label FMT patients; for Fusobacterium this involves either lack of eradication in patients who do not achieve remission, transplantation into patients without remission, or eradication in patients who achieve remission.

### Example 7. Treatment-Naïve Ulcerative Colitis Patient Treated with Oral Fecal Microbiome Therapy

A 44-year old treatment-naïve male patient (patient DM) presents with a one year history of diarrhea, blood and mucous in stool, cramping/abdominal pain, and weight loss. The patient experiences severe pain upon defecation, incontinence whilst driving, loss of appetite, nausea, inability to eat spicy foods and fish, brain fog, and weight loss of 16 lbs. The patient also experiences 10-12 bowel movements per day with a consistency of 7 (Bristol), severe bloating, severe abdominal discomfort, and severe urgency. The patient is diagnosed with severe pancolitis. A treatment regimen including acid resistant/delayed release double encapsulated oral capsules containing lyophilized donor-derived non-selected fecal microbiota is used. Briefly, donor stool is collected and homogenized with cryoprotectant and the resulting slurry is lyophilized and encapsulated in DRcaps® capsules containing ˜1.6×10¹¹ viable cells/capsule. The patient is treated with a total of 426 capsules over a 13-week induction period, followed by 724 capsules over a 29-week maintenance period (Total: 1150 capsules over a 42-week period). During this period, symptom questionnaires are collected and stools are cultured for pathogens to assess efficacy and safety of the treatment.

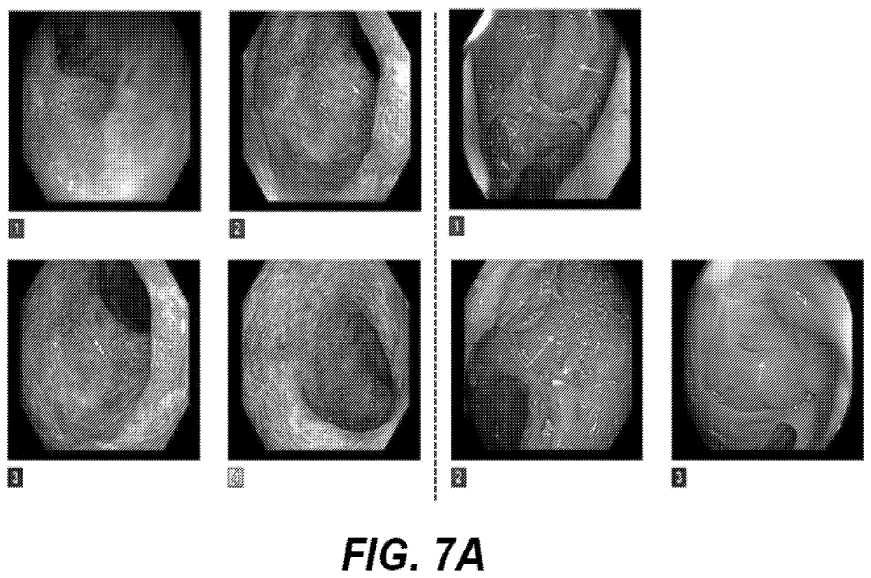

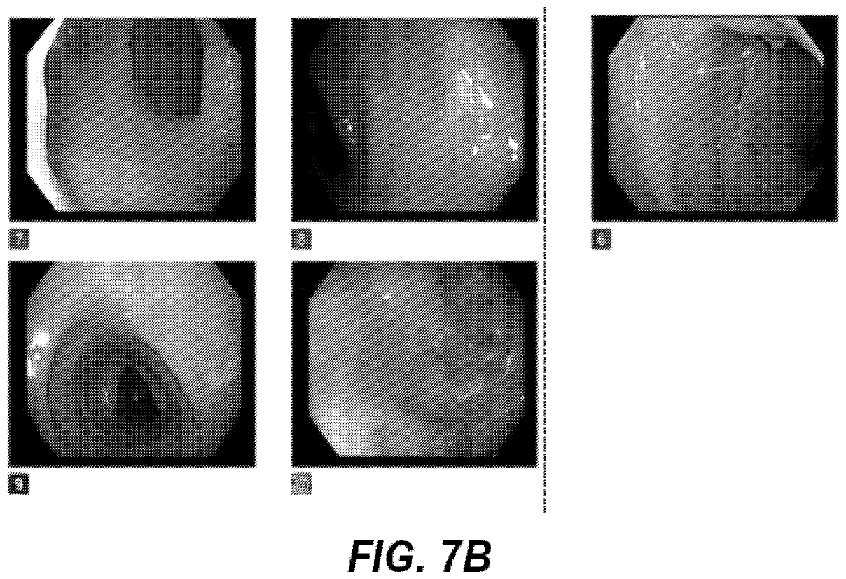

The patient's UC symptoms show improvement post-treatment (see Table 11). By week 8 post-treatment, blood and mucous are barely visible in stool, by week 10 it is nil. The patient's incontinence ceases and bowel motions decrease to 2-3/day with a consistency of 4 (Bristol). With continuing treatment, calprotectin levels decrease from 600 μg/g in week 22, to 344 μg/g in week 26, and eventually 50 μg/g in week 42. This confirms an ongoing inflammation reduction which is also shown by endoscopy in FIG. 7. The patient does not report any side-effects relating to the tolerability of the treatment. This case represents a successful treatment of ulcerative colitis (UC) with oral fecal microbiome therapy. In addition to the quality of life (QoL) improvement which result from the patient's UC symptom improvements, there is also a significant increase in energy levels that allows for daily exercise and confidence to recommence work due to reduced incontinence. The patient continues well on maintenance treatment of 4 capsules per day. Oral fecal microbiome therapy is efficacious when treating a treatment-naïve UC patient with pancolitis, resulting in an overall improved QoL.

TABLE 11
Comparison of symptoms prevalence at baseline and
post-treatment in case study patient.

| | | Week 26 | Week 42 |
|---|---|---|---|
| Symptoms | Baseline | Post-Treatment | Post-Treatment |
| Bowel Opening (per day) | 10-12 | 2-3 | 2-3 |
| Consistency (BSC) | 7 | 4-5 | 4 |
| Difficulty passing a motion | 2 | 1 | 1 |
| Pain/Abdominal discomfort | 3-4 | 1 | 1 |
| Urgency to pass a motion | 4 | 1 | 1 |
| Diarrhea | 4 | 1 | 1 |
| Blood in stool | 4 | 1 | 1 |
| Mucus in stool | 4 | 1-2 | 1 |
| General Malaise | 3-4 | 1-2 | 1 |

Consistency is measured as per Bristol Stool Chart (BSC).
Additional measurements: 1 = none; 2 = mild; 3 = moderate; 4 = moderately severe; 5 = severe.

### Example 8. Ulcerative Colitis Patient Treated with Oral Fecal Microbiome Therapy

A 31-year old patient (patient TD) is treated with fecal microbiome therapy. A treatment regimen including acid resistant/delayed release double encapsulated oral capsules containing lyophilized donor-derived non-selected fecal microbiota is used. The patient's symptoms include 4 bowel movements per day with a consistency of 2, moderate bloating, moderate abdominal discomfort, mild urgency, and feelings of pins and needles in legs and fatigue. The patient is placed on a 6 week treatment protocol with one fecal microbiome therapy liquid colonoscopic infusion and 1-2 rectal enema infusions per week during the induction period. The patient takes 4 capsules per day for 4 weeks during the maintenance period. The patient's symptoms of bloating decrease and symptoms of abdominal discomfort and urgency disappear. The patient experiences one bowel movement per day with a consistency of 3. The patient also experiences mild flatulence and general malaise. Two weeks after capsule treatment the patient has a calprotectin reading of 243 μg/g. Three weeks from the first calprotectin test and 4 weeks from the initial capsule intake, the patient's calprotectin level decreases to 88 μg/g.

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entirety.

1. A method of providing a therapeutic dosing regimen to a patient with a gastrointestinal (GI) disorder in need thereof, the method comprising administering to the patient a therapeutic composition comprising viable non-pathogenic fecal bacteria based upon a level of a fecal marker of intestinal inflammation, wherein said fecal marker comprises a protein secreted by an immune cell of said patient.

2. The method of claim 1, wherein the GI disorder is selected from the group consisting of Antibiotic Associated Colitis, Chronic Clostridium difficile Infection (CDI), Chronic constipation, Chronic Fatigue Syndrome (CFS), Collagenous Colitis, Colonic Polyps, Constipation Predominant FBD, Crohn's Disease, Functional Bowel Disease (FBD), Gastro-oesophageal Reflux, Irritable bowel syndrome (IBS) constipation-predominant, IBS diarrhea/constipation alternating, IBS diarrhea-predominant, IBS pain-predominant, Indeterminate Colitis, Inflammatory Bowel Disease (IBD), Microscopic Colitis, Mucous Colitis, Multiple Sclerosis, Non-ulcer Dyspepsia, Norwalk Viral Gastroenteritis, Pain Predominant FBD, Primary Clostridium difficile Infection (CDI), Primary Sclerosing Cholangitis (PSC), Pseudomembranous Colitis, Small Bowel Bacterial Overgrowth, Ulcerative Colitis, and Upper Abdominal FBD.

3. The method of claim 1, further comprising determining the level of a fecal marker for intestinal inflammation selected from the group consisting of calprotectin, lactoferrin, M2-PK, neopterin, metalloproteinases, myeloperoxidases, and polymorphonuclear elastase.

4. The method of claim 1, wherein said protein is selected from the group consisting of calprotectin, lactoferrin, M2-PK, neopterin, metalloproteinases, myeloperoxidases, and elastases.

5. The method of claim 1, wherein said therapeutic composition comprising viable non-pathogenic fecal bacteria comprises fecal microbiota from multiple donors.

6. The method of claim 5, wherein said fecal microbiota from multiple donors is blended.

7. The method of claim 1, wherein the method comprises increasing a dosage or a dosing frequency by at least 2 times for one to ten weeks when the patient exhibits a fecal calprotectin level of above 500 μg/g.

8. The method of claim 1, wherein the method comprises
a. gradually decreasing a dosage or a dosing frequency by at least about 20% for at least 2 weeks when the patient exhibits a fecal calprotectin level of below 500 μg/g; and
b. monitoring the fecal calprotectin level in the patient.

9. The method of claim 1, wherein said therapeutic composition is formulated as an enteric coated capsule or an acid-resistant capsule.

10. The method of claim 1, wherein the therapeutic dosing regimen comprises a dose from 10⁷ to 10¹⁴ cfu or total number of cells.

11. A method for optimizing the dosing regimen of a fecal microbe-based therapy in a patient with a gastrointestinal disorder in need thereof, the method comprising:
a. administering to the patient a therapeutic composition comprising fecal microbes at a first dosing regimen comprising a first dosage at a first dosing frequency;
b. determining the level of a fecal marker for intestinal inflammation in the patient, wherein said fecal marker comprises a protein secreted by an immune cell of said patient; and
c. modifying the first dosing frequency based on the level of the fecal marker for intestinal inflammation.

12. The method of claim 11, wherein the GI disorder is selected from the group consisting of Antibiotic Associated Colitis, Chronic Clostridium difficile Infection (CDI), Chronic constipation, Chronic Fatigue Syndrome (CFS), Collagenous Colitis, Colonic Polyps, Constipation Predominant FBD, Crohn's Disease, Functional Bowel Disease (FBD), Gastro-oesophageal Reflux, Irritable bowel syndrome (IBS) constipation-predominant, IBS diarrhea/constipation alternating, IBS diarrhea-predominant, IBS pain-predominant, Indeterminate Colitis, Inflammatory Bowel Disease (IBD), Microscopic Colitis, Mucous Colitis, Multiple Sclerosis, Non-ulcer Dyspepsia, Norwalk Viral Gastroenteritis, Pain Predominant FBD, Primary Clostridium difficile Infection (CDI), Primary Sclerosing Cholangitis (PSC), Pseudomembranous Colitis, Small Bowel Bacterial Overgrowth, Ulcerative Colitis, and Upper Abdominal FBD.

13. The method of claim 11, wherein the fecal marker for intestinal inflammation is selected from the group consisting of calprotectin, lactoferrin, M2-PK, neopterin, metalloproteinases, myeloperoxidases, and polymorphonuclear elastase.

14. The method of claim 10, wherein said protein is selected from the group consisting of calprotectin, lactoferrin, M2-PK, neopterin, metalloproteinases, myeloperoxidases, and elastases.

15. The method of claim 11, wherein said therapeutic composition comprising fecal microbes comprises fecal microbes from multiple donors.

16. The method of claim 15, wherein said fecal microbes from multiple donors are blended.

17. The method of any one of claim 1 or 11, wherein the fecal marker for intestinal inflammation is calprotectin.

18. The method of claim 13, wherein the method comprises
a. increasing the first dosage or the first dosing frequency by at least 2 times for one to ten weeks when the patient exhibits a fecal calprotectin level of above 500 μg/g; and
b. monitoring the fecal calprotectin level in the patient.

19. The method of claim 13, wherein the method comprises
a. gradually decreasing the first dosage or first dosing frequency by at least about 20% for at least 2 weeks when the patient exhibits a fecal calprotectin level of below 500 μg/g; and
b. monitoring the fecal calprotectin level in the patient.

20. The method of claim 18, wherein the method further comprising maintaining a stable dosing regimen in the patient when the patient exhibits a fecal calprotectin level of below 50 μg/g.

21. The method of claim 18, wherein the method comprises decreasing a dosage or a dosing frequency by at least about 20% for at least 2 weeks when the patient's fecal calprotectin level decrease by at least 20% from a baseline level.

22. The method of claim 11, wherein said therapeutic composition comprises live non-pathogenic fecal bacteria.

23. The method of claim 11, wherein the therapeutic dosing regimen comprises a dose from 10⁷ to 10¹⁴ cfu or total number of cells.

24. A method for selecting a fecal donor, the method comprising
a. administering to a test subject a fecal therapeutic composition comprising a substantially complete fecal microbiota derived from the fecal donor,
b. measuring a fecal marker for intestinal inflammation in the test subject, wherein said fecal marker comprises a protein secreted by an immune cell of said test subject,
c. selecting the fecal donor based on the level of the fecal marker for intestinal inflammation.

25. The method of claim 24, wherein the GI disorder is selected from the group consisting of Antibiotic Associated Colitis, Chronic Clostridium difficile Infection (CDI), Chronic constipation, Chronic Fatigue Syndrome (CFS), Collagenous Colitis, Colonic Polyps, Constipation Predominant FBD, Crohn's Disease, Functional Bowel Disease (FBD), Gastro-oesophageal Reflux, Irritable bowel syndrome (IBS) constipation-predominant, IBS diarrhea/constipation alternating, IBS diarrhea-predominant, IBS pain-predominant, Indeterminate Colitis, Inflammatory Bowel Disease (IBD), Microscopic Colitis, Mucous Colitis, Multiple Sclerosis, Non-ulcer Dyspepsia, Norwalk Viral Gastroenteritis, Pain Predominant FBD, Primary Clostridium difficile Infection (CDI), Primary Sclerosing Cholangitis (PSC), Pseudomembranous Colitis, Small Bowel Bacterial Overgrowth, Ulcerative Colitis, and Upper Abdominal FBD.

26. The method of claim 24, wherein the fecal marker for intestinal inflammation is selected from the group consisting of calprotectin, lactoferrin, M2-PK, neopterin, metalloproteinases, myeloperoxidases, and polymorphonuclear elastase.

27. The method of claim 24, wherein said protein is selected from the group consisting of calprotectin, lactoferrin, M2-PK, neopterin, metalloproteinases, myeloperoxidases, and elastases.

28. The method of claim 24, wherein said fecal therapeutic composition comprises substantially complete fecal microbiota derived from multiple donors.

29. The method of claim 28, wherein said substantially complete fecal microbiota from multiple donors is blended.
